(19) United States
(12) Patent Application Publication
(10) Pub. No.: US 2024/0091145 A1
(43) Pub. Date: Mar. 21, 2024

(54) DRY MUCO-ADHESIVE COMPOSITIONS AND USE THEREOF

(71) Applicants: TECHNION RESEARCH & DEVELOPMENT FOUNDATION LIMITED, Haifa (IL); Health Corporation Of Tzafon Medical Center (R.A.), Lower Galilee (IL)

(72) Inventors: Havazelet BIANCO-PELED, Haifa (IL); Avi SCHROEDER, Haifa (IL); Imad ABU EL-NAAJ, Nofit (IL); Layan HABIB, Haifa (IL); Mohammed ALYAN, Haifa (IL); Yasmin GHANTOUS, Shefa Amer (IL)

(21) Appl. No.: 18/527,308

(22) Filed: Dec. 3, 2023

Related U.S. Application Data
(63) Continuation of application No. PCT/IL2022/050591, filed on Jun. 2, 2022.
(60) Provisional application No. 63/196,355, filed on Jun. 3, 2021.

(51) Int. Cl. A61K 9/00; A61K 9/127

(52) U.S. Cl. CPC A61K 9/006; A61K 9/127; B82Y 5/00

(57) ABSTRACT

A composition and method for the administration of therapeutic and/or diagnostic agents is provided. Specifically, a dry hybrid system, composed of bio adhesive polymer that harbors drug-loaded lipid nanoparticles, and use thereof for the administration of active agents e.g., anti-cancer agents or biological agents, is provided.

### CROSS-REFERENCE

This application is a bypass continuation of PCT/IL2022/050591 filed Jun. 2, 2022, which claims the benefit of priority to U.S. Provisional Patent Application No. 63/196,355 filed Jun. 3, 2021 entitled “DRY MUCO-ADHESIVE COMPOSITIONS AND USE THEREOF”, the contents of which are incorporated herein by reference in their entirety.

### FIELD OF INVENTION

The present invention, in some embodiments thereof, is directed to compositions and methods for transmucosal delivery of agents, including but not limited to therapeutic agents, including but not limited to the oral cavity.

### BACKGROUND OF THE INVENTION

Transmucosal delivery involves transport of agents, including but not limited to therapeutic agents, through the mucosa; a moist gel layer that lines organs which are exposed on the outer surface of the body, yet are not covered with skin. This mode of delivery offers multiple benefits over oral or intravenous administration, especially when dealing with lesions of the oral cavity. For example, mucoadhesive drug delivery facilitates rapid circulation of drugs in the local capillaries. In addition, it enables enhanced bioavailability, resulting from the ability of mucoadhesive drug delivery to avoid some of the body's natural defense mechanisms and first-pass metabolism. Oral cavity cancer is the sixth most common cancer in the world. Oral Squamous Cell Carcinoma (OSCC) is the most diagnosed oral cancer and consider as part of the larger group of Head and Neck Squamous Carcinoma, accounting for approximately 550,000 new cases with two-thirds of the tumors diagnosed in developing countries and about 304,000 deaths worldwide annually. The tongue and the floor of the mouth are the most familiar sites correlates with a poor prognosis, reflected by 5-year and the 10-year survival rate of 60% and 48% respectively. Tobacco and alcohol consumption are the main risk factors linked to cancer of the oral cavity and pharynx, which are account for around 75% of all cases. Despite recent changes in surgical and oncological therapies for oral cancer, which involve aggressive treatments, the 5-year survival rates of patients have remained dismally low. Novel approaches for treating oral cancer based on drug delivery systems are constantly being developed to deal and improve the existing complications of the current surgical treatments. However, no such system of anti-cancer controlled drug delivery to the oral cavity, is available in the clinic. Developing a local drug delivery system is in a significant need to minimize the surgical intervention, hence decrease the need for reconstructive surgery and improve the long-term survival rate.

Drug delivery via the oral mucosa could serve as an alternative method and a promising route, owing to its both systemic action and local controlled therapy. The oral mucosa is highly vascularized which enable a trans-mucosal absorbance of therapeutic anti-cancer agents through mucus, a viscous liquid secretion that covers some skinless outer surfaces of the body, offering numerous benefits e.g. being a relatively painless administration method and having a higher bioavailability, between 4 and 4000 times more compared to the skin, due to the evasion of first pass metabolism, the gastrointestinal tract and natural barriers that defend the body. However, this method can face up some hindrances, e.g. a quick removal of the drug or the delivery system due to bulk salivary flow and the relatively low permeability of the buccal tissue. Mucoadhesive polymers can overcome these hindrances and generate a successful local drug delivery system by proficient attaching the dosage form for sufficient time on the oral mucosa, thanks to both physical and weak chemical bonds between some polymers and the mucin chains, the main component of mucus. The polymer's adhesive strength can vary depending on the molecular weight, cross-linking density, charge and ionization, concentration, pH or hydration degree of the polymer. Alginate, is a natural linear hydrophilic polysaccharide, consisting of two types of monomers, (1-4)-linked β-D-mannuronic acid (M) and α-L-guluronic acid (G) units and arranged randomly in M- and G-blocks. Alginate has been used in multiple biomedical applications, particularly as a drug delivery vehicle, owing to its biodegradable, non-irritant and biocompatible properties and having mucoadhesive properties due to its ability to form hydrogen bonds with the glycoprotiens through carboxyl-hydroxyl interactions.

There is an unmet need for compositions and methods for the transmucosal delivery of agents, e.g., therapeutic agents, such as in a sustained release manner. Further, for transmucosal delivery to the oral cavity, there is a need for compositions which are stable under dilution, shear flow and physiological conditions of the saliva fluids.

### SUMMARY OF THE INVENTION

The present invention provides compositions and methods useful for the transmucosal administration of therapeutic and/or diagnostic agents.

The present invention presents for the first time, inter alia, a dried matrix, composed of polymer (e.g., alginate) that harbors drug-loaded lipid nanoparticles, and use thereof for the administration of active agents e.g., anti-cancer agents for treating oral cancers.

In one aspect of the invention, there is provided a composition comprising a polymeric matrix and a plurality of nano-particles embedded within the polymeric matrix, wherein: (a) the polymeric matrix comprises a bio-adhesive polymer; (b) the plurality of nano-particles encapsulate a biologically active agent, (c) the composition comprises a cryoprotectant; (d) a water content of the composition is at most 5% by weight; and wherein a weight per weight (w/w) concentration of the a bio-adhesive polymer within the composition is at least 10%.

In one embodiment, at least 99.5% w/w of the bio-adhesive polymer is not-crosslinked.

In one embodiment, polymeric matrix is characterized by a network of interconnected chains of the bio-adhesive polymer.

In one embodiment, a w/w concentration of the bio-adhesive polymer within the composition is at most 95% w/w.

In one embodiment, a w/w ratio of the cryoprotectant to the plurality of nano-particles is between 2:1 and 20:1.

In one embodiment, a w/w ratio between the bio-adhesive polymer and the plurality of nano-particles is between 10:1 and 1:10.

In one embodiment, polymeric matrix is in a form of a layer, and wherein the plurality of nano-particles are homogenously distributed within the layer.

In one embodiment, composition comprises a plurality of layers.

In one embodiment, composition comprises an upper layer and a bottom layer comprising the polymeric matrix; and further comprises an intermediate layer comprising the plurality of nano-particles.

In one embodiment, bio-adhesive polymer comprises a mucoadhesive polymer selected from the group consisting of: alginate, chitosan, pectin, hyaluronic acid, PVA, and polyacrylate, including any salt, derivative, copolymer, or any combination thereof.

In one embodiment, plurality of nano-particles comprises a lipid and are in a form of liposomes or micelles.

In one embodiment, lipid comprises a phospholipid and a sterol.

In one embodiment, sterol comprises cholesterol, and the phospholipid comprises a zwitterionic lipid, an anionic lipid, a PEG-ylated lipid including any combination thereof.

In one embodiment, a w/w ratio between the phospholipid and the sterol is between 1:4 and 4:1.

In one embodiment, at least 80% of the plurality of nano-particles have an average diameter of between 50 and 500 nanometers.

In one embodiment, plurality of nano-particles is characterized by a polydispersity index of 0.03-0.3.

In one embodiment, the molar concentration of the plurality of nano-particles within the composition is about 10-200 millimolar (mM).

In one embodiment, cryoprotectant is selected from the group consisting of a disaccharide, DMSO, a glycol, glycerol, or any combination thereof.

In one embodiment, a w/w concentration of said cryoprotectant within said composition is between 0.1 and 10%.

In one embodiment, upon reconstitution of the composition the average diameter of the plurality of nano-particles increases by at most 30%.

In one embodiment, upon contact with a mucous tissue the composition is capable of releasing at least 50% of the biologically active agent.

In one embodiment, releasing is within a time period of between 0.1 and 24 h.

In another aspect, there is provided an article comprising the composition of the invention, and wherein the article is a bio-adhesive article, optionally characterized by mucoadhesiveness.

In one embodiment, the article comprises an effective amount of the biologically active agent, wherein the biologically active agent comprises any one of: a pharmaceutical agent, a nutraceutical agent, a taste masking agent, a flavoring agent.

In one embodiment, article is in a form of a film, and wherein the article further comprises an additional non-mucoadhesive layer.

In one embodiment, a thickness of the article is between 0.1 and 10 mm, and any one of a length dimension and of a width dimension of the article is between 1 mm and 10 cm.

In one embodiment, biologically active agent is characterized by having a therapeutic effect in the treatment of an oral cavity disease.

In one embodiment, mucoadhesiveness comprises stress at maximum load of at least 2 KPa.

In another aspect, there is provided a method for preventing or treating a medical condition, comprising administering the article of the invention to a subject, thereby preventing or treating the medical condition.

In one embodiment, administering comprising contacting the composition with a biological tissue of the subject.

In one embodiment, biological tissue comprises a mucous tissue, a dermal tissue, a muscle tissue, and a urinary bladder tissue or any combination thereof.

In one embodiment, administering is selected from the group consisting of oral administration, nasal administration, and dermal administration, or any combination thereof.

In one embodiment, oral administration comprises buccal administration, sublingual administration or both.

In one embodiment, medical condition comprises an oral cavity disorder.

In another aspect, there is provided a method of manufacturing the article of the invention, comprising: a. exposing an aqueous solution comprising the plurality of nano-particles and the cryoprotectant to conditions sufficient for drying of the aqueous solution, thereby obtaining a dry powder; b. performing any one of (i) or (ii):
(i) mixing a sufficient amount of the dry powder with a powderous composition comprising the muco-adhesive polymer, thereby obtaining a mixture;
(ii) applying a compression force to a powderous composition comprising a bio-adhesive polymer, thereby shaping a polymeric layer; applying said dry powder on top of said polymeric layer, thereby obtaining an intermediate layer; and applying said polymeric layer on top of said intermediate layer, thereby obtaining a layered composition; and c. applying a compression force to said mixture or to said layered composition, thereby manufacturing the article.

In one embodiment, conditions sufficient for drying comprises lyophilization.

In one embodiment, bio-adhesive polymer comprises a mucoadhesive polymer selected from the group consisting of: alginate, chitosan, pectin, hyaluronic acid, PVA, and polyacrylate, including any salt, derivative, copolymer, or any combination thereof.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C are graphs representing Dynamic light scattering (DLS) measurements of the particle size of liposomes before and after freeze-drying (1A) without adding cryoprotectant, (1B) with 9:1 (w/w) trehalose:lipids and (1C) with alginate as cryoprotectant.

FIGS. 2A-2C are micrographs representing cryogenic scanning electron microscope (Cryo-SEM) images of (2A) liposome-trehalose solution before freeze-drying, using Cryo-SEM, scale bar 100 nm, (2B) side view of layered tablet containing trehalose, using SEM, scale bar 100 nm, and (2C) freeze-dried and rehydrated liposomes without cryoprotectant, using Cryo-SEM, scale bar 101 μm. White arrows points to liposomes and red arrows points to footprints of liposomes.

FIG. 3 is a graph representing fractional release of Doxorubicin (Dox) from freeze-dried liposomes and as-prepared liposomes in simulated saliva buffer vs. time, pH=6.8 at 37° C.

FIGS. 4A-4D are graphs representing Fourier transform infrared spectroscopy (FTIR) spectra of (4A) freeze-dried empty liposomes, trehalose powder and freeze-dried trehalose-liposomes solution, (4B) freeze-dried empty liposomes, alginate powder, freeze-dried alginate-liposomes mixture, (4C) as-prepared liposomes with different amounts of trehalose added as trehalose:lipid mass ratio, and (4D) showing magnification of the FTIR spectrum presented in FIG. 4C.

FIGS. 5A-5B are graphs representing fractional release of rhodamine labelled liposomes from (5A) homogeneous tablets and (5B) layered tablets containing different amounts of alginate vs. time in simulated saliva buffer, pH=6.8 at 37° C.

FIGS. 6A-6B are schematic representation of exemplary articles of the invention: multi-layered tablet (6A), single layer homogeneous tablet (6B).

FIGS. 7A-7B are graphs representing fractional release of Dox from single layer homogeneous tablet (7A) and form multi-layered tablet (7B) containing different amounts of alginate presented as mass ratio, vs. time in simulated saliva buffer, pH=6.8 at 37° C.

FIG. 8 is a graph representing stress at maximum load vs. tablets containing different alginate amount. Statistical differences are only depicted in comparison to the 0% alginate tablet.

FIGS. 9A-9B are graphs representing mucoadhesion evaluation of tablets containing diverse alginate-FITC amounts using flow through experiment in simulated saliva buffer, pH=6.8 at 37° C. FIG. 9A is a comparison between the adhesion performance of homogeneous tablets and layered tablets with the same alginate amount (layered: green and orange graphs; Homogenous: magenta and blue graphs). FIG. 9B is a comparison between the adhesion performance of homogeneous tablets with different alginate mass ratios.

FIGS. 10A-10D are bar graphs representing cell viability of SCC7 cell line in the presence of single layer homogeneous and multi-layered tablets with different amounts of alginate, (10A) 43% (w/w) alginate, (10B) 57% (w/w) alginate, short term, (10C) 43% (w/w) alginate, (10D) 57% (w/w) alginate, long term. *p<0.05, **p<0.005, ***p<0.0005, ****p<0.0001.

FIGS. 11A-11D are images representing tablet insertion in tongues in homogenous tablet. 11A: incision is made across tongue. 11B: Tablet implant. 11C: Wound sealing using medical glue. 11D: Healing of incision wound after 24 hrs.

FIGS. 12A-12B are images representing tumor development in homogenous tablet. 12A: Tongue visualization in non-treated group. 12B: Tongue visualization in treated group.

FIGS. 13A-13F are images representing tablet insertion in tongues in layered tablet. 13A: protruded tumor on tongue. 13B: Placement of layered tablet above tumor. 13C: Addition of 20 ul PBS to adhere the layered tablet on tumor. 13D: Tongue after removal of layered tablet. Tumor development in layered tablet, non-treated group (13E) and in treated group (13F).

FIG. 14 is an image representing tongue visualization upon different treatment at ×2 magnification.

FIGS. 15A-15C are graphs showing body weight loss after treatment with layered and homogenous tablets (15A), tumor size after treatment with layered and homogenous tablets (15B), and tumor size 10 days post treatment with layered and homogenous tablets (15C).

FIGS. 16A-16D are CT images of mice at first (0) and last (10) day of treatment with a layered tablet loaded with Dox (16D) and a homogenous tablet loaded with Dox (16C), as compared to a tablet without Dox (16B) and to untreated mice (16A). *p<0.05, **p<0.005, ***p<0.0005.

FIGS. 17A-17E are Histological images of tongues. (17A) Healthy, (17B) No-treatment, (17C) Empty, (17D) Homogenous tablet, (17E) Layered tablet. Images are shown in two magnifications, right and left columns with ×1.2 and ×40, respectively.

### DETAILED DESCRIPTION OF THE INVENTION

The present invention in some embodiments thereof, is directed to compositions and methods useful for transmucosal delivery of at least one active agent (e.g., a therapeutic agent, a nutraceutical agent, a diagnostic agent or any combinations thereof).

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

According to an aspect of some embodiments of the present invention, there is provided a dry composition comprising a bio-adhesive polymer and a nano-carrier, wherein the nano-carrier is encapsulated by or embedded within the bio-adhesive polymer, and wherein the nano-carrier comprises at least one agent selected from a therapeutic agent and a diagnostic agent.

In some embodiments, the term “bio-adhesive” refers to a feature of a substance (e.g., a formulation or a matrix) having improved adhesiveness to a biological tissue. In some embodiments, the bio-adhesive polymer comprises a muco-adhesive polymer. In some embodiments, the term “mucoadhesive” refers to a substance capable of adherence to a mucus tissue. In some embodiments, the mucoadhesive composition is capable of stably attaching to the mucus, thus prolonging the residence time of the formulation at the application site.

### Composition

In one aspect of the invention, provided herein a composition comprising a polymeric matrix and a plurality of nano-particles embedded within the polymeric matrix, wherein: (a) the polymeric matrix comprises a bio-adhesive polymer; (b) the plurality of nano-particles encapsulate a biologically active agent, (c) the composition comprises a cryoprotectant; (d) a water content of the composition is at most 5% by weight; and wherein a weight per weight (w/w) concentration of the bio-adhesive polymer within the composition is at least 10%.

In another aspect of the invention, there is provided a composition comprising a polymeric matrix and a plurality of nano-particles embedded within the polymeric matrix, wherein: (a) the polymeric matrix comprises a bio-adhesive polymer; (b) the plurality of nano-particles encapsulate a biologically active agent, (c) a water content of the composition is at most 5% by weight; and wherein a weight per weight (w/w) concentration of the a bio-adhesive polymer within the composition is at least 10%. In some embodiments, the composition is a dry composition. In some embodiments, the composition is a powderous composition. In some embodiments, the composition is a bioadhesive composition. In some embodiments, the composition is a mucoadhesive composition.

As used herein the term “bioadhesive composition” refers to composition or a part thereof (e.g. of the bioadhesive polymer) characterized by adhesiveness to a biological tissue. As used herein, the term “biological tissue” refers to any tissue comprising inter alia epithelial tissue and/or mucous tissue or a cancer tissue. In some embodiments, the composition of the invention and/or the bioadhesive polymer is characterized by adhesiveness to a cancer tissue.

In some embodiments, the water content of the composition of the invention is at most 5%, at most 3%, at most 2%, at most 1%, at most 0.5%, at most 0.3%, at most 0.1%, at most 0.01% by total weight of the composition.

In another aspect of the invention, there is provided a composition comprising a polymeric matrix and a plurality of nano-particles embedded within the polymeric matrix, wherein: (a) the polymeric matrix comprises a bio-adhesive polymer; (b) the plurality of nano-particles encapsulate a biologically active agent, (c) a water content of the composition is at most 5% by weight; and wherein at least 99.5% w/w of the bio-adhesive polymer is not crosslinked.

In some embodiments, the bio-adhesive polymer is a non-crosslinked polymer. In some embodiments, at least 95%, at least 97%, at least 99%, at least 99.1%, at least 99.5%, at least 99.9%, by total weight of the bio-adhesive polymer is non-crosslinked, including any range between.

As used herein the term “crosslinked” refers to polymeric chains which are inter-connected (crosslinked) by ions, such as divalent metal cations.

In some embodiments, the bio-adhesive polymer is or comprises a muco-adhesive polymer. In some embodiments, the disclosed composition comprises a polymeric material (e.g. bio-adhesive or muco-adhesive polymer) in a form of a bio-adhesive (e.g. muco-adhesive) matrix, characterized by a network of intertwisted polymeric chins. In some embodiments, the network is further characterized by internal pores. In some embodiments, one or more nano-particles of the invention are entrapped or embedded within the network e.g., within the internal pores.

In some embodiments, an average diameter of the internal pores is at least 5%, at least 10%, at least 20%, or at least 30% smaller than an average or a median diameter of the one or more nano-carriers.

In some embodiments, the composition of the invention comprises at least 5%, at least 10%, at least 15%, at least 20%, at least 25%, at least 30%, at least 40%, at least 50%, by weight of the bio-adhesive polymer, including any range between. In some embodiments, the composition of the invention comprises at most 80%, at most 85%, at most 90%, at most 92%, at most 95%, at most 97%, by weight of the bio-adhesive polymer, including any range between.

The bio-adhesive polymers used in the present invention may be a copolymer with one or more other monomers unless they do not undesirable effect on the physio-chemical properties of the polymers, and include also a polymer crosslinked by an appropriate crosslinking agent. When the bio-adhesive polymers are copolymerized with not more than 30% by mole of the other monomer(s), the physio-chemical properties of the polymers are not affected, and hence, such copolymers can be used in the present invention.

In some embodiments, the bio-adhesive polymer is hydrophilic or water-swellable polymeric material comprising a polysaccharide, a poly-amino acid, or both including any derivate, and/or salt thereof. In some embodiments, the bio-adhesive polymer comprises a charged polymer, including any salt and/or any derivative thereof (e.g. a copolymer, or a chemically modified polymeric chain). In some embodiments, the bio-adhesive polymer comprises a positively charged polymer and/or a negatively charged polymer. In some embodiments, the bio-adhesive polymer comprises an ionizable polymer. In some embodiments, the bio-adhesive polymer comprises an ionizable polysaccharide.

For example, in some embodiments, the bio-adhesive polymer is selected from, without being limited thereto, pectin, chitosan, alginate, cellulose, hyaluronic acid, PVA, polyacrylate, polyacrylate ester (e.g. PMMA) or any derivative and/or any salt thereof.

In some embodiments, the composition of the invention is substantially biodegradable and/or bioerodible. In some embodiments, the composition of the invention is biocompatible.

In some embodiments, at least 80%, at least 90%, at least 95%, at least 97%, at least 99%, at least 99.1%, at least 99.5%, at least 99.9%, of the composition of the invention is biodegradable and/or bioerodible, including any range between In some embodiments, at least 80%, at least 90%, at least 95%, at least 97%, at least 99%, at least 99.1%, at least 99.5%, at least 99.9%, of the composition of the invention is biodegradable and/or bioerodible within a time period ranging from 1 to 72 h, from 1 to 3 h, from 3 to 5 h, from 5 to 7 h, from 7 to 10 h, from 10 to 12 h, from 12 to 24 h, from 24 to 48 h, from 48 to 72 h, including any range between.

In some embodiments, the bio-adhesive polymer is a biodegradable and/or bioerodible polymer. The term “biodegradable” describes a substance which can decompose under physiological and/or environmental conditions into breakdown products. Such physiological and/or environmental conditions include, for example, hydrolysis (decomposition via hydrolytic cleavage), enzymatic catalysis (enzymatic degradation), and mechanical interactions. This term typically refers to substances that decompose under these conditions such that e.g., 50 weight percent of the substance decompose within a time period shorter than one year.

Furthermore, as used herein, the term “biodegradable”, is intended to describe materials comprising covalent bonds that are degraded in vivo, wherein the degradation of the covalent bond occurs via hydrolysis. The hydrolysis can involve a direct reaction with an aqueous medium or can be catalyzed chemically or enzymatically. “Aqueous medium” refers to water, aqueous solutions, physiological media or biological fluids (e.g., body fluids), and other pharmaceutically acceptable media. Suitable hydrolysable covalent bonds are selected from the group containing: esters, amides, urethanes, carbamates, carbonates, ethers, azo linkages, anhydrides, thioesters, and combinations thereof.

The term “biodegradable” as used in the context of embodiments of the invention, also encompasses the term “bioresorbable”, which describes a substance that decomposes under physiological conditions to break down products that undergo bioresorption into the host-organism, namely, become metabolites of the biochemical systems of the host-organism. In some embodiments, the term biodegradable as used in the context of embodiments of the invention refers to the ability of a material to undergo substantial dissolution, e.g. gradual dissolution within the time period as described herein.

In some embodiments, the mucoadhesive polymer is at least partially biodegradable. In some embodiments, the mucoadhesive polymer is fully biocompatible. In some embodiments, the mucoadhesive polymer is biodegradable and biocompatible.

As used herein, the term “biocompatible”, is intended to describe materials that, are non-toxic to cells in vitro and upon administration in vivo, do not induce undesirable long-term effects.

In some embodiments, the bio-adhesive polymer of the invention may comprise a combination of bio-stable polymers and/or biodegradable polymers. In some embodiments, the bio-adhesive polymer is biocompatible.

In some embodiments, the bio-adhesive polymer is a mucoadhesive polymer. In some embodiments, the mucoadhesive polymer is capable of stably binding or adhering to mucosa. In some embodiments, the mucoadhesive polymer is capable of stably binding or adhering to a mucous tissue of a subject in need thereof. In some embodiments, the mucoadhesive polymer is characterized by a desired friction and/or hydrophilicity so as to enable binding or adherence to mucosa, for example by formation of inter-chain bridges of the polymeric functional group and mucin glycoproteins.

In some embodiments, the composition or article of the invention refers herein as stably bound to mucosa, if the article and/or the composition substantially maintains its adhesiveness to the biological tissue. In some embodiments, the stably bound or adhered composition or article is applicable to a target site of the subject, and maintains its structural and/or mechanical integrity at the target site for at least 1 h, at least 2 h, at least 3 h, at least 5 h, including any range between. In some embodiments, the stably bound or adhered composition or article is substantially (e.g. at least 60%, at least 70%, at least 80%, at least 90%, at least 95%, by weight of the composition and/or article, including any range between) retained at the application site. In some embodiments, the composition or article is substantially retained at the application site for at least 1 h, at least 2 h, at least 3 h, at least 5 h, at least 10 h, at least 24 h, at least 48 h, at least 72 h, including any range between, after applying thereof to the application site.

In some embodiments, the application site is a biological tissue. In some embodiments, the application site is an outer surface of the biological tissue. In some embodiments, the application site is an interior of the biological tissue. In some embodiments, the application site is an outer surface and/or interior of the mucous tissue.

In some embodiments, the composition or article is substantially retained on top or within the biological tissue (e.g. implanted) for at least 1 h, at least 2 h, at least 3 h, at least 5 h, at least 10 h, at least 24 h, at least 48 h, at least 72 h including any range between.

In some embodiments, the bio-adhesive polymer or mucoadhesive polymer disclosed herein is or comprises alginate, including any derivative (e.g. alginic acid, a copolymer of alginic acid), copolymer and/or any salt thereof.

In some embodiments, the mucoadhesive polymer is characterized by an average molecular weight ranging from 5000 to 10.000 Da, from 10000 Da to 900,000 Da, from 10000 Da to 100,000 Da, from 10,000 Da to 50,000 Da, from 50,000 Da to 100,000 Da, from 100,000 Da to 200,000 Da, from 200,000 Da to 300,000 Da, from 300,000 Da to 400,000 Da, from 400,000 Da to 500,000 Da, from 500,000 Da to 600,000 Da, from 600,000 Da to 900,000 Da, including any range or value therebetween.

In some embodiments, the composition comprises a polymeric matrix. In some embodiments, the composition comprises and/or is formed by the mucoadhesive polymer of the invention. As used herein, the term “polymeric matrix” refers to one or more polymeric layer. Matrix may further include any materials incorporated within and/or interposed between the layers.

In some embodiments, the polymeric matrix is a multi-layer matrix, comprising a mucoadhesive layer and an additional layer. In some embodiments, at least 70%, at least 80%, at least 90%, at least 95%, at least 99%, at least 99.9% by weight of the polymeric matrix consists of the mucoadhesive polymer.

As used herein, the term “polymer”, or any grammatical derivative thereof, describes an organic substance composed of a plurality of repeating structural units (monomeric units) covalently connected to one another.

In some embodiments, the bioadhesive polymer (e.g. mucoadhesive polymer) of the invention is or comprises pharmaceutically acceptable polymer or pharmaceutically acceptable salt thereof.

### Nano-Particles

In some embodiments, the composition of the present invention provides a bioadhesive (e.g. muco-adhesive) delivery vehicle for any one of a drug and/or a diagnostic agent (herein “agent”). In some embodiments, the composition of the present invention comprises vesicles or carriers, such as nano-particles (e.g., liposomes or micelles) encapsulated within a polymer having one or more muco-adhesive groups or regions. The nano-particles can be loaded by an agent, or the agent can be otherwise carried by the nano-particles. The incorporation of the agent within the vehicle may be by any means, whether in the interior or on the exterior of the membrane of the vesicle.

In some embodiments, the nano-particles are in contact with the bioadhesive polymer (e.g. in a form of a polymeric matrix). In some embodiments, the nano-particles are dispersed within or incorporated into the polymeric matrix of the invention. In some embodiments, the nano-particles are embedded within the polymeric matrix of the invention. In some embodiments, the nano-particles are bound to the bioadhesive polymer. In some embodiments, the nano-particles are encapsulated by the bioadhesive polymer. In some embodiments, the nano-particles are homogenously mixed with the bioadhesive polymer within the composition of the invention. In some embodiments, the nano-particles are uniformly distributed within the polymeric matrix. In some embodiments, the nano-particles are in a form of a layer in contact with or encapsulated by the bioadhesive polymer (e.g. in a form of a layer). In some embodiments, the nano-particles are in a form of an intermediate layer flanked by one or more layers of the bioadhesive polymer.

In some embodiments, the nano-particles are distributed in the bioadhesive polymer (e.g. in a form of a single-layered polymeric matrix) substantially uniformly in a single liposome form or in the form of clusters of liposomes. In some embodiments, the liposomes within the composition are held physically, or electrostatically, through non-covalent bonds in the polymeric matrix.

In some embodiments, the composition is in a form of a layer. In some embodiments, the layer is a single layer or a plurality of layers. In some embodiments, the composition comprises a single layer polymeric matrix or a multi-layered polymeric matrix. In some embodiments, the multi-layered matrix comprises a plurality of polymeric layers. In some embodiments, the multi-layered matrix comprises layers having the same or different compositions. In some embodiments, the multi-layered matrix comprises a plurality of distinct layers.

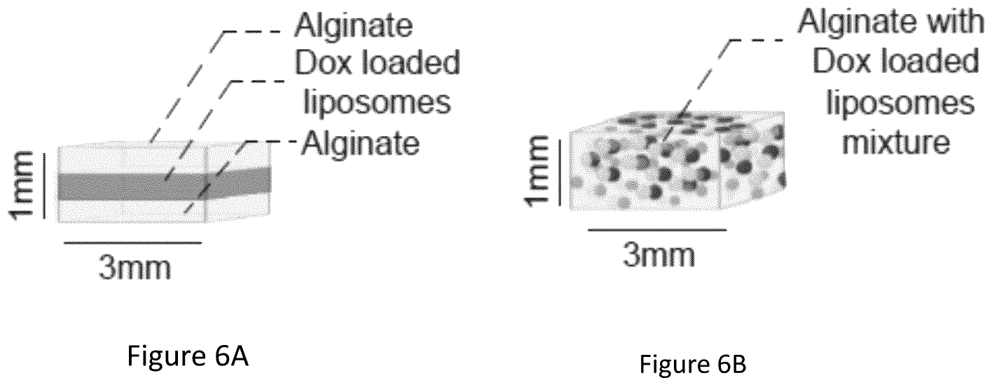

In some embodiments, the composition comprises one or more layers. In some embodiments, the composition comprises a single layer, comprising the bioadhesive polymer of the invention (e.g. in a form of a polymeric matrix) and nano-particles homogenously dispersed within or incorporated into the bioadhesive polymer (see FIG. 6B).

In some embodiments, the composition comprises a plurality of layers, comprising subsequent layers in contact or bound to each other. In some embodiments, the composition comprises a first layer comprising the bioadhesive polymer (e.g. in a form of a polymeric matrix) bound to a second layer comprising the nano-particles. In some embodiments, the composition comprises an upper layer and a bottom layer comprising the bioadhesive polymer (e.g. in a form of a polymeric matrix); and further comprises an intermediate layer comprising the plurality of nano-particles, and wherein the upper layer and the bottom layer are bound to the intermediate layer (see FIG. 6A).

In some embodiments, the layered composition is stable, e.g. being devoid of disintegration under prolonged storage (ranging from 1 month to 5 years, including any range between) at ambient conditions.

In some embodiments, the at least one nano-particle is in a form of a vesicle, wherein the vesicle forms a complex/particulate with the carried materials (e.g. biologically active agent) with or without an additional agent such as a polymer, protein, or salt. In some embodiments, the at least one nano-particle forms a dendrimer like structure, in which the components of the dendrimer like structure are conjugated to the polymeric backbone or complexed via van der Waals or hydrophobic interactions.

In some embodiments, “vesicle” and “carrier” are synonymous and refer to a particle (e.g. the nano-particle of the invention) comprising a core and a shell encapsulating or enclosing the core. In some embodiments, the nano-particle of the invention comprises a core and a shell encapsulating or enclosing the core. In some embodiments, the core is a hollow core, or a core filled with a material. In some embodiments, the core comprises a biologically active agent, substantially located therewithin. In some embodiments, the membrane comprises one or more layers. In some embodiments, the membrane comprises a bi-layer. In some embodiments, the active agent is bound to the membrane. In some embodiments, the active agent is located between the membrane layers. In some embodiments, the active agent is located within the membrane (e.g. within the bi-layer). In some embodiments, the nano-particle of the invention may have a spherical or any other geometrical shape. In some embodiments, the nano-particle of the invention comprises a unilamellar or multilamellar membrane. In some embodiments, the nano-particle of the invention comprises one or more different types of nano-particles. In some embodiments, “by different types” it is meant to refer to liposomes that encapsulate different active agents (e.g., drugs). In some embodiments, “by different types” it is meant to refer to liposomes that are of different structure and configurations.

In some embodiments, the nano-particle is configured for delivery of polynucleotide, such as antisense oligonucleotide, or of RNA. In some embodiments, the particle comprises a plurality of lamellae. In some embodiments, the nanoparticle is a unilamellar or a multilamellar nanoparticle. In some embodiments, the polynucleotide is bound to a plurality of lamellae so as to form a polyplex, wherein the polyplex. In some embodiments, the nanoparticle comprises or is a polyplex. In some embodiments, the polyplex comprises a polynucleotide in contact with or bound to the amphiphilic polymer. In some embodiments, the polynucleotide is bound to the amphiphilic polymer via a non-covalent bond. In some embodiments, the polynucleotide is bound to the amphiphilic polymer via an electrostatic interaction. In some embodiments, the amphiphilic polymer comprises a polyamino acid. In some embodiments, the amphiphilic polymer is or comprises positively charged residues. In some embodiments, the amphiphilic polymer is or comprises a cationic polymer.

In some embodiments, the concentration of the nano-particle of the invention is in a range from about 20 mM to 200 mM, or from about 30 mM to 150 mM, or from about 40 mM to 120 mM, from about 50 mM to 100 mM, including any value and range therebetween.

In some embodiments, the nano-particle of the invention has a size (or diameter) in the range of about 50 to 500 nanometers (nm). In some embodiments, the composition of the invention comprises a plurality of nano-particles, wherein the size or diameter of nano-particles refers to an average or to a median size or diameter.

In some embodiments, the size of the nano-particles of the invention is in a range of between 50 and 300 nm, between 50 and 100 nm, between 100 and 150 nm, between 150 and 200 nm, between 200 and 250 nm, between 250 and 300 nm, including any value and range therebetween. In some embodiments, at least 80%, at least 85%, at least 90%, at least 95%, at least 97% by weight of the nano-particles of the invention has a particle size in a range of between 50 and 300 nm, between 50 and 100 nm, between 100 and 150 nm, between 150 and 200 nm, between 200 and 250 nm, between 250 and 300 nm, including any value and range therebetween. In some embodiments, at least 80%, at least 85%, at least 90%, at least 95%, at least 97% by weight of the nano-particles of the invention has a particle size in a range of between 50 and 150 nm.

In some embodiments, the median size of the nano-particles within the composition (e.g. dispersed throughout the bulk of the mucoadhesive polymer), is about 50 nm, about 60 nm, about 70 nm, about 80 nm, about 90 nm, about 100 nm, about 150 nm, about 200 nm, about 250 nm, about 300 nm, about 400 nm, about 500 nm, in diameter, including any value and range therebetween.

In some embodiments, the nano-particles of the invention are characterized by a polydispersity index of between 0.03 and 0.3, between 0.03 and 0.05, between 0.05 and 0.1, between 0.1 and 0.15, between 0.15 and 0.2, between 0.2 and 0.3, including any value and range therebetween. In some embodiments, the nano-particles of the invention are characterized by a median size, as described hereinabove, and are further characterized by polydispersity index of between 0.03 and 0.3, between 0.03 and 0.05, between 0.05 and 0.1, between 0.1 and 0.15, between 0.15 and 0.2, between 0.2 and 0.3, including any value and range therebetween.

In some embodiments, the size and/or size distribution of the nano-particles of the invention may be measured by any method known in the art (e.g., Dynamic Light Scattering; DLS or by SEM/cryo-SEM, etc.). In some embodiments, the size and/or size distribution refers to the size of the dry nano-particles within the composition of the invention and/or upon reconstitution thereof. The reconstituted size can be measured inter alia by DLS.

In some embodiments, the size of the nano-particles (e.g. liposomes) described herein refers to the size which has been measured shortly before incorporation and/or drying of the liposomes. In some embodiments, the liposomes are stable, e.g. do not substantially change their size once entrapped into the bioadhesive polymer of the invention.

Unstable nano-particles (e.g. liposomes) are disadvantageous for the instantly disclosed invention, wherein unstable refer to nano-particles which don't retain its shape and/or size and/or decompose upon drying, so as to release the biologically active agent therefrom.

In some embodiments, the nano-particle of the invention is or comprises a lipid-based particle. In some embodiments, the nano-particle of the invention is or comprises a liposome. In some embodiments, liposomes refer to vesicles with an internal core surrounded by a lipid bilayer/s, and are widely used as drug carriers. This is greatly due to their unique characteristics such as good biocompatibility, low toxicity, lack of immune system activation, and the ability to incorporate both hydrophobic and hydrophilic compounds. As described herein, liposomes are known in the art as artificial vesicles composed of a substantially spherical lipid bilayer which typically, but not exclusively, comprises phospholipids, sterol, e.g., cholesterol, and other lipids.

Hereinthroughout, “liposomes” refer to one or more liposomes.

In some embodiments, the liposomes are characterized by a proper packing parameter. As used herein and in the art, packing parameter is a relative measure of a given lipid composition, and depend on factors such as size relationships between lipid head groups and lipid hydrocarbon chains, charge, and the presence of stabilizers such as cholesterol. It should also be noted that the packing parameter may be not constant. In some embodiments, the parameter is dependent on various conditions which effect each the volume of the hydrophobic chain, the cross-sectional area of the hydrophilic head group, and the length of the hydrophobic chain. Factors can affect these include, but are not limited to, the properties of the solvent, the solvent temperature, and the ionic strength of the solvent.

In some embodiments, the proper packing parameter is in the range of 0.3 to 1, e.g., 0.3, 0.5, 0.7, 0.9, or 1, including any value and range therebetween.

In some embodiments, the liposome is characterized by a desired surface charge, anionic surface charge, or cationic surface charge, as described hereinbelow.

Without being bound by any particular theory, it is noted herein that the use of liposomes dispersed in the bioadhesive polymer rather than the use of liposome in liquid suspension, is advantageous, as it provides a reservoir of liposomes within the bioadhesive polymer (or a polymeric matrix) that provide a desired release profile (e.g., controlled release) of the liposomes and substances encapsulated therein from the disclosed composition to a targeted area.

In some embodiments, the nano-particle of the invention is substantially dry. In some embodiments, the nano-particle of the invention is a dried particle (e.g. a freeze dried particle). In some embodiments, the nano-particle of the invention is characterized by a water content of at most 5%, at most 3%, at most 2%, at most 1%, at most 0.5%, at most 0.3%, at most 0.1%, at most 0.01% by total weight of the nano-particles.

In some embodiments, a molar ratio of the nano-particles within the composition is between 10-200 mM, between 10-50 mM, between 50-100 mM, between 100-200 mM, including any range between.

In some embodiments, a w/w concentration of the nano-particles within the composition is between 5 and 50%, between 5 and 10%, between 10 and 20%, between 20 and 30%, between 30 and 50%, including any range between.

In some embodiments, a w/w ratio of the nano-particles to the bioadhesive polymer within the composition is between 50:1 and 1:2, between 50:1 and 40:1, between 40:1 and 30:1, between 30:1 and 20:1, between 20:1 and 10:1, between 10:1 and 1:10, between 10:1 and 8:1, between 8:1 and 6:1, between 6:1 and 4:1, between 4:1 and 2:1, between 2:1 and 1:1, between 1:1 and 1:2, between 1:2 and 1:4, between 1:4 and 1:8, between 1:8 and 1:10, including any range between.

In some embodiments, the nano-particles are comprised of at least one phospholipid and/or a sterol (e.g. cholesterol and/or a derivative thereof).

In some embodiments, the at least one phospholipid is or comprises a cationic lipid. In some embodiments, the at least one phospholipid is or comprises a non-cationic lipid. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. In some embodiments, the at least one phospholipid comprises a non-cationic lipid and/or optionally comprises a cationic lipid.

Non-cationic lipids include, but are not limited to, phosphatidylcholine (HSPC), distearoyl-phosphatidyl-ethanolamine (DSPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), 16-O-monomethyl PE, 16-O-dimethyl PE, 18-1-trans PE, 1-stearoyl-2-oleoyl-phosphatidyethanolamine (SOPE), 2-dimyristoyl-sn-glycero-3-phosphocholine, hydrogenated soybean phosphatidylcholine, or a mixture thereof. Such non-cationic lipids may be used alone, or used in combination with other excipients, for example, cationic lipids. When used in combination with a cationic lipid, the non-cationic lipid may comprise a molar ratio of 5% to about 90%, or about 10% to about 70% of the total lipid present in the nano-particle of the invention. The invention contemplates the use of lipid nanoparticles as transfer vehicles comprising a cationic lipid to encapsulate and/or enhance the delivery of nucleic acid into the target cell. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available.

Suitable cationic lipids for use in the compositions and methods of the invention include those described in international patent publication WO 2010/053572, incorporated herein by reference.

In some embodiments, the at least one phospholipid is or comprises a PEG-ylated lipid. In some embodiments, the PEG-ylated lipid comprises a polyethyleneglycol (PEG) moiety covalently bound to the lipid molecule, and/or to a derivative thereof. In some embodiments, the PEG-ylated lipid comprises a PEG-modified lipid. In some embodiments, the terms “PEG-ylated lipid” and “PEG-modified lipid” are used herein interchangeably. In some embodiments, the PEG-ylated lipid comprises PEG-moiety covalently bound to the phosphate group of the lipid. In some embodiments, the PEG-moiety is covalently bound to the amine group via one or more linkers, such as C1-C10alkyl linker, or any other linker or functional group capable of covalently binding the PEG moiety to the lipid. In some embodiments, the PEG-moiety is covalently bound to the amine group (e.g. ethanolamine group of the lipid) via a carbonyl group. In some embodiments, the PEG-moiety is covalently bound to the lipid via an amide group. In some embodiments, the PEG-moiety comprises PEG and/or a derivative thereof, e.g. a PEG modified with an alkyl (e.g. methyl) or an alkyl derivative at the terminal end of the PEG chain. In some embodiments, the PEG-moiety comprises a methylated PEG (m-PEG).

In some embodiments, the PEG-moiety is characterized by a molecular weight (MW) ranging between 100 and 10.000 Da, between 100 and 500 Da, between 500 and 1000 Da, between 1000 and 1500 Da, between 1500 and 2000 Da, between 2000 and 2500 Da, between 2500 and 3000 Da, between 3000 and 5000 Da, between 5000 and 10.000 Da, including any range between.

Using of PEG-ylated lipids may be beneficial for stability of the nano-particle of the invention after and during the drying process, and may also allow homogenization of the nano-particle within the matrix, thus preventing aggregation thereof. Furthermore, PEG moieties may also enhance circulation lifetime of the nano-particle in-vivo, or they may be selected to enhance release of the nano-particles from the polymeric matrix and/or the composition of the invention under in-vivo conditions.

In some embodiments, by “homogenization” it is meant that the concentration of at least 10%, at least 20%, at least 30%, at least 40%, at least 50%, or at least 60% of the volume of the nano-particles within the polymeric matrix varies within less than ±20%.

In some embodiments, the at least one phospholipid comprises a phospholidpid (e.g. a non-cationic lipid, such as HSPC) and a PEG-ylated lipid. In some embodiments, the at least one phospholipid comprises HSPC and/or DSPE. In some embodiments, the at least one phospholipid comprises HSPC and DSPE-PEG. In some embodiments, a w/w ratio between the phospholidpid and the PEG-ylated lipid within the nano-particle of the invention is between 10:1 and 1:1, between 10:1 and 6:1, between 6:1 and 4:1, between 4:1 and 2:1, between 2:1 and 1:1, including any range between.

In some embodiments, a molar ratio between the phospholidpid and the PEG-ylated lipid within the nano-particle of the invention is between 20:1 and 3:1, between 20:1 and 15:1, between 15:1 and 12:1, between 12:1 and 10:1, between 10:1 and 8:1, between 8:1 and 5:1, between 5:1 and 3:1, including any range between.

In some embodiments, a w/w ratio between the phospholipid (e.g. a non-PEG-yalted lipid, such as HSPC and optionally a PEG-yalted lipid, such as DSPE-PEG) and sterol (e.g. cholesterol) within the nano-particle of the invention is between 5:1 and 1:5, between 5:1 and 4:1, between 4:1 and 3:1, between 3:1 and 1:1, between 2:1 and 1:1, between 1:1 and 1:3, between 1:3 and 1:5, including any range between.

In some embodiments, a molar ratio between the phospholipid (e.g. a non-PEG-yalted lipid, such as HSPC and optionally a PEG-yalted lipid, such as DSPE-PEG) and sterol (e.g. cholesterol) within the nano-particle of the invention is between 2:1 and 1:2, between 2:1 and 1:1, between 1:1 and 1:2, including any range between.

The selection of lipids, non-cationic lipids and/or PEG-modified lipids which comprise the nanoparticle of the invention, as well as the relative molar ratio of such lipids to each other, is based upon the characteristics of the selected lipid(s), the nature of the intended target cells and the characteristics of the agents to be delivered. Additional considerations include, for example, the saturation of the alkyl chain, as well as the size, charge, pH, pKa, fusogenicity and toxicity of the selected lipid(s).

The liposomal transfer vehicles for use in the compositions of the invention can be prepared by various techniques which are presently known in the art. Multi-lamellar vesicles (MLV) may be prepared conventional techniques, for example, by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Uni-lamellar vesicles (ULV) can then be formed by homogenization, sonication or extrusion of the multi-lamellar vesicles. In addition, unilamellar vesicles can be formed by detergent removal techniques.

In some embodiments, the nano-particle (e.g. liposome) of the invention comprises HSPC, PEG-DSPE and cholesterol in a defined ratio. In some embodiments, the liposome of the invention comprises HSPC:PEG-DSPE:cholesterol in a ratio of from about 50:10:40 to 55:5:40 or from 60:2:38 to 50:10:40, respectively including any value and range therebetween. In certain embodiments, the liposome of the invention comprises HSPC:PEG-DSPE:cholesterol in the ratio of about 55:5:40, respectively.

In some embodiments, the nano-particle (e.g. liposome) of the invention is composed of the pharmaceutically acceptable ingredients (such as phospholipids and/or sterol) or pharmaceutically acceptable salt thereof.

### Therapeutic and Diagnostic Agents

In some embodiments, the nano-particle of the invention encapsulates the biologically active agent. In some embodiments, the nano-particle of the invention is formulated to deliver one or more biologically active agent to one or more target cells. In some embodiments, the nano-particle of the invention allows the encapsulated biologically active agents to reach the target cell and/or may preferentially allow the encapsulated agents to reach the target cell, or alternatively limit the delivery of the agents to other undesired target sites or cells. In some embodiments, the nano-particle of the invention enhances performance of the biologically active agent by improving solubility and bioavailability thereof, in vitro and in vivo stability, as well as preventing unwanted interactions of the biologically active agent with other molecules. Another advantage of the nano-particle of the invention is cell-specific targeting, which is a prerequisite to attain drug concentrations required for optimum therapeutic efficacy in the target cell while minimizing adverse effects on healthy cells and tissues.

In some embodiments, the nano-particles of the invention encapsulate an effective amount (e.g. therapeutically effective amount) of the biologically active agent. In some embodiments, the nano-particles of the invention are characterized by a loading of the biologically active agent (also refers to herein, as the drug loading) sufficient for utilizing thereof in the treatment or prevention of a disease. In some embodiments, the biologically active agent comprises a pharmaceutically active agent (e.g. a drug) and/or a diagnostic agent (e.g., a labeling agent). In some embodiments, the biologically active agent is attached to and/or encapsulated within the nano-particle (e.g., liposome). In some embodiments, the composition of the invention comprises an effective amount (e.g. therapeutically effective amount) of the biologically active agent. In some embodiments, the composition of the invention is a pharmaceutical composition comprising a therapeutically effective amount of the biologically active agent. In some embodiments, the composition of the invention is a pharmaceutical composition comprising a therapeutically effective amount of the nano-particles of the invention.

As used herein, the term “a therapeutically active agent” describes a chemical substance, which exhibit a therapeutic activity when administered to a subject. As used herein, the term “biologically active agent”, or “bioactive agent”, describes a chemical or a biological substance, which exhibits a biological or physiological activity in an organism.

As used herein, a “therapeutically effective amount” or “an amount effective” refers to an amount effective, at dosages and for periods of time necessary, to achieve the desired therapeutic result. The therapeutically effective amount of the therapeutic agent will depend on the nature of the disorder or condition and on the particular agent and can be determined by standard clinical techniques known to a person skilled in the art.

As used herein, the term “labeling agent” refers to a detectable moiety or a probe and includes, for example, chromophores, fluorescent compounds, phosphorescent compounds, heavy metal clusters, and radioactive labeling compounds, as well as any other known detectable moieties. Also included are contrast agents, e.g., a magnetic resonance imaging (MM) contrast agent, a computed tomography (CT) contrast agent, a single photon emission computed tomography (SPECT) contrast agent, a positron emission tomography (PET) contrast agent, a bioluminescence (BL) contrast agent, an optical contrast agent, an X-ray contrast agent, and an ultrasonic contrast agent.

The term “radioactive agent” describes a substance (i.e. radionuclide or radioisotope) which loses energy (decays) by emitting ionizing particles and radiation. When the substance decays, its presence can be determined by detecting the radiation emitted by it. For these purposes, a particularly useful type of radioactive decay is positron emission. Exemplary radioactive agents include ^{99m}Tc, ¹⁸F, ⁶⁷Ga, ¹³¹I and ¹²⁵I.

In some embodiments, the biologically active agent is a hydrophobic and/or a hydrophilic agent. In some embodiments, the biologically active agent comprises a medicament suitable for treating a disease.

Non-limiting examples of therapeutically active agents that can be beneficially used in embodiments of the present invention include, without limitation, one or more of an anti-inflammatory drug, an anti-proliferative drug, polynucleotide, an antisense oligonucleotide, RNA (e.g. oligo RNA, siRNA, micro-RNA, mRNA and modified RNA), DNA, a chemotherapeutic drug, a terpene, a cannabinoid, an agonist agent, an amino acid agent, an analgesic agent, an antagonist agent, an antibiotic agent, an antibody agent, an antidepressant agent, an antigen agent, an antihistamine agent, an anti-hypertensive agent, an anti-metabolic agent, an antimicrobial agent, an antioxidant agent, a radical (or ROS) scavenging agent, a co-factor, a cytokine, a drug, an enzyme, a growth factor, a heparin, a hormone, an immunoglobulin, an inhibitor, a ligand, a nucleic acid, an oligonucleotide, a peptide, a phospholipid, a prostaglandin, a protein, a toxin, a vitamin and any combination thereof. In some embodiments, the biologically active agent comprises a radical (or ROS) scavenging agent, specifically one or more ionizing radiation protecting agents (e.g. ascorbic acid, cinnamic acid, polyphenols, polyunsaturated compounds, carotenoids, etc.).

In some embodiments, the polynucleotide comprises a plurality of polynucleotide types. In some embodiments, the nanoparticle comprises a plurality of polynucleotide types. In some embodiments, the composition comprises a plurality of nanoparticle types, each type of nanoparticle comprises a specific polynucleotide.

In some embodiments, a polynucleotide comprises RNA, DNA, a synthetic analog of RNA, a synthetic analog of DNA, DNA/RNA hybrid, or any combination thereof. In some embodiments, a nanoparticle of the invention comprises a polynucleotide selected from: RNA, DNA, a synthetic analog of RNA, a synthetic analog of DNA, DNA/RNA hybrid, or any combination thereof.

In some embodiments, the biologically active agent comprises a therapeutic agent for treatment of one or more mucous related disease. In some embodiments, the biologically active agent comprises a therapeutic agent for treatment of one or more diseases or conditions selected from oral diseases (e.g. oral cancer), teeth diseases and also systemic diseases. In some embodiments, the biologically active agent comprises a cannabinoid (e.g. CBD, THC or any derivative thereof). In some embodiments, the biologically active agent comprises a therapeutic agent such as anti-viral agents, anti-proliferative agent, analgesics and anti-inflammatory agents (e.g. indomethacin, ibuprofen), mouth disinfectants or anti-fungal agents, (e.g. chlorohexidine hydrochloride, hexylresorcinol), anti-ulcer agent, enzymes (e.g. lysozyme chloride, dextranase, kallikrein), coronary vasodilators (e.g. nitroglycerin, isosorbide dinitrate, nifedipine), antiasthmatics (e.g. di sodium cromoglycate), antibiotics (e.g. penicillins, erythromycin), chemotherapeutics (e.g. sulfathiazole, nitrofurazone), local anesthetics (e.g. benzocaine), cardiotonics (e.g. digitalis, digoxin), antitussives and expectorants (e.g. codeine phosphate, isoproterenol hydrochloride), agents affecting digestive organs (e.g. water-soluble azulene (sodium azulene sulfonate), vitamin U), antihistamines (e.g. diphenhydramine hydrochloride, chlorpheniramine maleate), anti-inflammatory steroids (e.g. prednisolone, triamcinolone), antifungal agents (e.g., miconazole, nystatin and amphotericin), hemostatics, sexual hormones, sedatives, antitumor agents, or the like.

In some embodiments, the therapeutically active agent is an agent for treatment of aphthae. In some embodiments, the therapeutically active agent is an anti-cancer drug. The term “cancer” as used herein, refers to a disease or disorder resulting from the proliferation of ontogenically transformed cells. Examples of particular cancers that may be treated according to the method of the present invention include oral cancer, such as oral squamous cell carcinoma and oral pharyngeal cancer.

The phrase “anticancer agent” or “anticancer drug”, as used herein, describes a therapeutically active agent that directly or indirectly kills cancer cells or directly or indirectly inhibits, stops or reduces the proliferation of cancer cells. Anti-cancer agents include those that result in cell death and those that inhibit cell growth, proliferation and/or differentiation. In some embodiments, the anti-cancer agent is selectively toxic against certain types of cancer cells but does not affect or is less effective against normal cells. In some embodiments, the anti-cancer agent is a cytotoxic agent.

Examples of cancer therapeutic agents include, e.g., but are not limited to Abiraterone, Acitretin, Aldesleukin, Alemtuzumab, Amifostine, Amsacrine, Anagrelide, Anastrozole, Arsenic, Asparaginase, Asparaginase Erwinia, Axitinib, azaCITItidine, BCG, Bendamustine, Bevacizumab, Bexarotene, Bicalutamide, Bleomycin, Bortezomib, Brentuximab, Bromocriptine, Buserelin, Busulfan, Cabazitaxel, Cabergoline, Capecitabine, CARBOplatin, Carmustine, Cetuximab, Chlorambucil, ClSplatin, Cladribine, Clodronate, Crizotinib, Cyclophosphamide, CycloSPORINE, Cytarabine, Dacarbazine, Dactinomycin, Dasatinib, DAUNOrubicin, Degarelix, Denosumab, Dexamethasone, Dexrazoxane, DOCEtaxel, DOXOrubicin, DOXOrubicin pegylated liposomal, Enzalutamide, Epirubicin, Eribulin, Erlotinib, Estramustine, Etoposide, Everolimus, Exemestane, Filgrastim, Fludarabine, Fluorouracil, Flutamide, Fulvestrant, Gefitinib, Gemcitabine, Goserelin, Hydroxyurea, IDArubicin, Ifosfamide, Imatinib, Iniparib, Interferon alfa-2b, Ipilimumab, Irinotecan, Ixabepilone, Lambrolizumab, Lanreotide, Lapatinib, Lenalidomide, Letrozole, Leucovorin, Leuprolide, Lomustine, Mechlorethamine, medroxyPROGESTERone, Megestrol, Melphalan, Mercaptopurine, Mesna, Methotrexate, mitoMYCIN, Mitotane, mitoXANTRONE, Nilotinib, Nilutamide, Octreotide, Ofatumumab, Oxaliplatin, PACLitaxel, ACLitaxel nanoparticle, albumin-bound (nab), Pamidronate, Panitumumab, Pazopanib Pemetrexed, Pertuzumab, Porfimer, Procarbazine, Quinagolide, Raltitrexed, Reovirus Serotype 3-Dearing Strain, riTUXimab, Romidepsin, Ruxolitinib, SORAfenib, Streptozocin, SUNItinib, Tamoxifen, Temozolomide, Temsirolimus, Teniposide, Testosterone, Thalidomide, Thioguanine, Thiotepa, Thyrotropin alfa, Tocilizumab, Topotecan, Trastuzumab (HERCEPTIN®), Trastuzumab, Emtansine (KADCYLA®), Treosulfan, Tretinoin, Vemurafenib, vinBLAstine, vinCRIstine and Vinorelbine.

Examples of chemotherapeutic agents used as a therapeutic agent include, e.g., but are not limited to, e.g., alkylating agents (e.g., cyclophosphamide, ifosfamide, melphalan, chlorambucil, aziridines, epoxides, alkyl sulfonates), cisplatin and its analogues (e.g., carboplatin, oxaliplatin), antimetabolitites (e.g., methotrexate, 5-fluorouracil, capecitabine, cytarabine, gemcitabine, fludarabine), toposiomerase interactive agents (e.g., camptothecin, irinotecan, topotecan, etoposide, teniposide, doxorubicin, daunorubicin), antimicrotubule agents (e.g., vinca alkaloids, such as vincristine, vinblastine, and vinorelbine; taxanes, such as paclitaxel and docetaxel), interferons, interleukin-2, histone deacetylase inhibitors, monoclonal antibodies, estrogen modulators (e.g., tamoxifen, toremifene, raloxifene), megestrol, aromatase inhibitors (e.g., letrozole, anastrozole, exemestane, octreotide), octreotide, anti-androgens (e.g., flutamide, casodex), kinase and tyrosine inhibitors (e.g., imatinib (STI571 or Gleevac); gefitinib (Iressa); and erlotinib (Tarceva), etc. See, e.g. Cancer: Principles and Practice of Oncology, 7th Edition, Devita et al, Lippincott Williams & Wilkins, 2005, Chapters 15, 16, 17, and 63).

In some embodiments, the biologically active agent is a nutraceutical (e.g. a vitamin, an antioxidant, a phytosterol, an unsaturated fatty acid, a plant extract, or a combination thereof)

In some embodiments, the biologically active agent is or comprises a taste masking agent (e.g. a sweetener, and/or any other excipient), a flavoring agent, a deodorizing agent, etc.

### Cryoprotectant

In some embodiments, the nano-particles of the invention are further bound or in contact with a cryoprotectant. In some embodiments, the cryoprotectant is capable of stabilizing (e.g. substantially retaining the size and/or the loading of the biologically active agent within the nano-particles) the nano-particles throughout and after the drying process. In some embodiments, the nano-particles substantially retain its geometrical shape and/or size upon drying thereof (e.g. by lyophilization). In some embodiments, the cryoprotectant facilitates stabilization of the nano-particles of the invention throughout and after the drying process. In some embodiments, the cryoprotectant facilitates retention of the initial loading of the biologically active agent within the nano-particles throughout and after the drying process.

In some embodiments, the nano-particles of the invention bound or in contact with a cryoprotectant are embedded within the mucoadhesive polymeric matrix of the invention.

In some embodiments, the nano-particles of the invention bound or in contact with a cryoprotectant substantially retain the initial loading of the biologically active agent and/or initial particle size upon drying of the nano-particles. In some embodiments, initial loading or initial particle size refer to loading or to particle size of the nano-particles before drying. In some embodiments, the nano-particles of the invention bound or in contact with a cryoprotectant substantially retain its intactness (geometrical shape, physical properties, size, loading, or a combination thereof) upon drying of the nano-particles. In some embodiments, the nano-particles of the invention bound or in contact with a cryoprotectant substantially retain its function as nano-carriers (e.g. capable of delivering a biologically active agent to a target site). In some embodiments, the nano-particles of the invention bound or in contact with a cryoprotectant remain intact upon drying and subsequent reconstitution thereof. In some embodiments, the nano-particles of the invention bound or in contact with a cryoprotectant are substantially devoid of disintegration or aggregation upon drying thereof.

The inventors facilitated formation of liposomes embedded within the mucoadhesive polymeric matrix and configured to stably encapsulate the active agent, by utilizing trehalose as the cryoprotectant. The inventors observed that compositions being devoid of a cryoprotectant (such as alginate and/or trehalose) are characterized by altered average dimeter and size distribution of the liposomes. Furthermore, it was found that compositions being devoid of the cryoprotectant were characterized by a substantial drug leakage during lyophilization and/or after reconstitution of the dry composition.

In some embodiments, the composition or article of the invention comprises an amount of the cryoprotectant sufficient for facilitating a substantial retention of the initial loading or initial particle size, upon drying of the nano-particles.

In some embodiments, a w/w ratio of the cryoprotectant to the plurality of nano-particles within the composition of the invention is between 2:1 and 20:1, between 2:1 and 5:1, between 5:1 and 10:1, between 10:1 and 15:1, between 15:1 and 20:1, including any range between.

In some embodiments, a w/w ratio of the cryoprotectant to the plurality of nano-particles within the composition is at least 2:1, at least 5:1, at least 9:1, at least 15:1, including any range between. In some embodiments, a w/w ratio of the cryoprotectant to the plurality of nano-particles within the dry composition of the invention is so, that upon reconstitution of the dry composition the average diameter of the nano-particles increases by at most 5%, at most 10%, at most 20%, at most 30% including any range between.

In some embodiments, a w/w concentration of the cryoprotectant within the composition is between 0.1 and 10%, between 0.1 and 1%, between 1 and 3%, between 3 and 5%, between 5 and 10%, including any range between.

In some embodiments, the cryoprotectant is or comprises alginate. In some embodiments, the composition of the invention comprises alginate as a cryoprotectant and is substantially devoid of an additional cryoprotectant.

In some embodiments, the cryoprotectant is a disaccharide (such as sucrose, or trehalose). In some embodiments, the cryoprotectant is trehalose. In some embodiments, the composition of the invention comprises trehalose as a cryoprotectant. Other cryoprotectants are known in the art, such as DMSO, a glycol (e.g. ethylene glycol), and glycerol.

### Sustained-Release Formulations

In another embodiment, provided herein the composition of the invention or an article comprising thereof, wherein the composition or article is capable of releasing the biologically active agent therefrom, upon contact with a mucous tissue; and wherein the composition and/or article is characterized by bio-adhesiveness. In some embodiments, the release is gradual or sustained release. In some embodiments, the composition or article disclosed herein is a sustained-release preparation characteristic in that it is easily adhered to mucous membrane in oral cavity and the adhesion is substantially maintained for a time period ranging, for example, between 0.1 and 24 hours.

In some embodiments, the article of the invention is substantially stable (e.g. maintains at least 60%, at least 80%, at least 90% of its geometrical shape, physical structure and/or physical or chemical properties, is substantially devoid of disintegration, and/or being capable of releasing the biologically active agent in a sustained manner, or a combination thereof) at the application site for a time period ranging, for example, between 0.1 and 24 hours. In some embodiments, the time period is predetermined by the chemical composition and/or configuration of the article. Besides, it can be kept within the oral cavity without being peeled off even by usual mouth action such as drinking, smoking, eating and speaking.

In some embodiments, the article of the invention is a mucoadhesive article, characterized by stress at maximum load of at least 2 KPa, at least 5 KPa, at least 10 KPa, at least 15 KPa, at least 150 KPa, including any range between. Mucoadhesiveness of the article can be determined as described in the Examples section.

In some embodiments, the release rate of the nano-particles (e.g. liposomes) from the article is dependent on the degradation profile or erosion rate (e.g. dissolution) of the polymeric matrix or of the article. In some embodiments, the release rate of the nano-particles is dependent on the diffusion rate of the nano-particles in the polymeric matrix and/or on the pore size of the polymeric matrix.

The release rate of the active agent from the polymeric matrix of the invention is governed inter alia by the chemical composition of the polymer chains, the concertation of the polymer/nano-particles of the invention, physical properties (such as density, pore size, homogenous or layered configuration of the article), and by the physical conditions at the application site.

In some embodiments, the article is a bio-adhesive article characterized by adhesiveness to a biological tissue. In some embodiments, the article is a mucoadhesive sustain-release article, characterized by adhesiveness to a mucous tissue.

The combined mucoadhesiveness and therapeutic effect of the composition and/or article disclosed herein is particularly advantageous when the composition or article is used in an application that also requires a localized enhanced effect of the therapeutically active agent.

In some embodiments, combining mucoadhesiveness with the advantages of liposomal drug delivery, such as a sustained release rate, allows protecting pharmaceuticals from chemical and enzymatic degradation, and improving drug bioavailability, hence providing a powerful method for non-invasive hybrid (polymer/lipid) drug delivery vehicles.

In some embodiments, the article or the composition is configured for delivery of the biologically active to an area of the body having a mucous membrane, such as, but not limited to, the oral cavity. For example, the delivery vehicle can be designed for use in oral, buccal, nasal, gastrointestinal, intratracheobronchial, pulmonary, rectal and vaginal routes for both systemic and local effects.

In some embodiments, the article of the invention is configured to release the nano-particles dispersed or embedded therewithin, upon contacting of the article with a mucous tissue. In some embodiments, the article of the invention is configured for release of the biologically active agent enclosed therewithin, upon contacting of the article with a mucous tissue. In some embodiments, the article of the invention is configured for release of the biologically active agent enclosed therewithin, upon reconstitution thereof, and wherein upon reconstitution the average diameter of the nano-particles increases by at most 5%, at most 10%, at most 20%, at most 30% including any range between.

In some embodiments, the article of the invention is configured to release at least 10%, at least 30%, at least 50%, at least 60%, at least 70%, at least 80%, at least 90%, at least 95% of the nano-particles including any range between. In some embodiments, the article of the invention is configured to release at least at least 10%, at least 30%, 50%, at least 60%, at least 70%, at least 80%, at least 90%, at least 95% of the biologically active agent enclosed therewithin, including any range between.

In some embodiments, the article of the invention is configured to release an effective amount of the biologically active agent. In some embodiments, the article of the invention is configured to release an effective amount of the nutraceutical and/or of an excipient (e.g. a flavoring agent), sufficient for supplementing a subject with the nutraceutical and/or excipient. In some embodiments, the article of the invention is configured to release a therapeutically effective amount of the pharmaceutically active agent, sufficient for the treatment of a disease or a disorder in a subject in need thereof.

In some embodiments, the article of the invention is configured to release the nano-particles and/or the biologically active agent upon contacting of the article with a mucous tissue. In some embodiments, the article of the invention is configured to release the nano-particles and/or the biologically active agent within a predetermined time period. In some embodiments, the exact length of the predetermined time period may vary, dependent on the application (e.g. treated disease, dose, and severity of the disease.

In some embodiments, the article of the invention is characterized by a prolonged release time of the biologically active agent, compared to a control. In some embodiments, prolonged comprise at least 20%, at least 50%, at least 60%, at least 70%, at least 80%, at least 90%, at least 95%, at least 97%, at least 99%, at least 200%, at least 300%, at least 400%, at least 500% greater release time, compared to a control. In some embodiments, the control comprises a similar article being devoid of the bio-adhesive polymer of the invention.

In some embodiments, the predetermined time period is between 0.1 and 48 h, between 0.1 and 1 h, between 1 and 3 h, between 3 and 5 h, between 5 and 10 h, between 10 and 15 h, between 15 and 20 h, between 20 and 25 h, between 25 and 30 h, between 30 and 48 h, including any range between.

In some embodiments, the release rate of the biologically active agent form the article of the invention is controllable by modifying the composition and or physical configuration (e.g. layering, density, etc.) of the article of the invention. In some embodiments, the time period for release of at least 50% by weight of the biologically active agent is controllable by modifying the composition and or physical configuration (e.g. layering, density, etc.) of the article of the invention. In some embodiments, the time period for release of at least 50% by weight of the biologically active agent is controllable by adding an additional polymeric layer on top of the article of the invention, wherein the additional layer is as described herein.

In some embodiments, the article of the invention is swellable. In some embodiments, the article of the invention is swellable upon contact thereof with water and/or with mucus. In some embodiments, the article of the invention is in a form of a layer, a tablet, or a film (e.g. dry film). In some embodiments, the article of the invention is in a form of a swellable layer, a swellable tablet, or a swellable film. In some embodiments, the article of the invention comprises at least one outer mucoadhesive layer. In some embodiments, the outer mucoadhesive layer is configured for application at the mucous tissue. In some embodiments, the outer mucoadhesive layer is configured for application at the oral cavity. In some embodiments, the outer mucoadhesive layer and/or the article is characterized by a sufficient mucoadhesiveness and by any of: mechanical strength, elasticity, shapeability, stretchability, Young's modulus compatible with the application on the mucus (e.g. within the oral cavity).

In some embodiments, the article of the invention comprises a single (homogenous) layer, or a plurality of layers. In some embodiments, the article of the invention comprises two subsequent layers bound to each other, wherein the first layer comprises the polymer of the invention and the additional layer comprises the nano-particles of the invention. In some embodiments, the article of the invention comprises a first homogenous layer, comprising the nano-particles uniformly distributed within the polymeric matrix; and at least one additional layer. In some embodiments, the additional layer is as described herein.

In some embodiments, the article of the invention comprises three subsequent layers bound to each other, wherein the first layer and the third layer are substantially identical outer mucoadhesive layers and comprise the polymeric matrix of the invention, and the intermediate layer comprises the nano-particles of the invention, and wherein the subsequent layers are stably bound to each other. In some embodiments, the intermediate layer is partially or completely enclosed by the outer mucoadhesive layer. In some embodiments, the first layer and the third layer are different layers.

In some embodiments, the article of the invention comprises (i) a first mucoadhesive outer layer comprising the bio-adhesive polymer of the invention, and configured for attachment to the mucus; and (ii) an additional outer layer configured to face an ambient (e.g. the oral cavity). In some embodiments, the additional layer as described herein is in a form of a polymeric layer. In some embodiments, the additional layer is substantially devoid of bio-adhesiveness and/or mucoadhesiveness. In some embodiments, the additional layer comprises a cross-linked polymer. In some embodiments, the additional layer comprises a cross-linked bio-adhesive polymer of the invention.

In some embodiments, the term “cross-linked” refers to a plurality of intra- and/or inter-molecular linkages forming a mesh-like polymeric scaffold or matrix. In some embodiments, the intra- and/or inter-molecular linkages are formed via one or more cross-linking agent(s).

In some embodiments, the polymeric chains within the additional layer are inter-connected (crosslinked) by one or more cross-linking agent(s). In some embodiments, the cross-linking agent is selected from covalent crosslinking agents, coordinative cross-linking agents (e.g. boric acid), electrostatic crosslinking agents (or ionic crosslinkers). In some embodiments, the cross-linking agent comprises an ion (e.g. divalent cations, such as calcium-, barium-, and/or strontium cations). Other non-limiting examples of crosslinkers (e.g., for alginate) are selected from sodium tripolyphosphate, phosphorus oxychloride or carboxylic acids. In some embodiments, the polymeric chains within the additional layer are inter-connected by a pharmaceutically acceptable cross-linkers and/or salts thereof. In some embodiments, the additional layer provides a barrier configured to prevent leakage of the nano-particles of the invention from the article, such as into the oral cavity.

In some embodiments, the additional layer is in a barrier layer, configured for preventing undesired adhesion of the article. In some embodiments, the additional layer is configured for preventing adsorption of particles (e.g. dust, moisture, or any other contamination) on top of the article. In some embodiments, the additional layer comprises a packaging material. In some embodiments, the additional layer comprises a polymeric material. In some embodiments, the additional layer comprises a thermoplastic polymer.

In some embodiments, the cross-linking degree of the polymeric chains within the additional layer is so as to prevent a release or leakage of the nano-particles of the invention (e.g. liposomes) throughout the additional layer.

In some embodiments, the additional layer comprises a polymer is selected from, without being limited thereto polyvinyl alcohol, polyethylene glycol, and polypropylene vinyl pyrrolidone, polytetrafluoroethylene, a fluorinated polyolefin, polyvinyl fluoride, polyethylene terephthalate (PET), polycyclohexylenedimethylene terephthalate, polycyclohexylenedimethylene terephthalate (PCTG), polyethylene naphthalate (PEN), polytrimethylene terephthalate (PTT), polybutylene terephthalate (PBT), a polyether, a polyolefinpolycarbonate, polycaprolactone (PCL), polylactic acid (PLA), polylactic-co-glycolic acid (PLGA), polyhydroxyethyl methacrylate (polyHEMA), and polyurethane, including any combination or a copolymer thereof.

In some embodiments, the article of the invention is characterized by a thickness between 0.1 and 10 mm, from 1 to 10 μm, from 10 to 20 μm, from 20 to 30 μm, from 30 to 40 μm, from 40 to 50 μm, from 50 to 100 μm, from 100 to 150 μm, from 150 to 200 μm, from 200 to 300 μm, from 300 to 400 μm, from 400 to 500 μm, from 500 to 600 μm, from 600 to 700 μm, from 700 to 800 μm, from 800 to 900 μm, from 900 to 1000 μm, from 1000 to 2000 μm, from 2000 to 3000 μm, from 3000 to 4000 μm, from 4000 to 5000 μm, from 5000 to 10.000 μm, including any range or value therebetween.

In some embodiments, the terms “thick” or “thickness” including any grammatical form thereof, refer to an average thickness.

In some embodiments, the article of the invention is characterized by any one of a length dimension and of a width dimension ranging between 1 mm and 10 cm, between 1 mm and 5 mm, between 5 mm and 1 cm, between 1 and 5 cm, between 5 and 10 cm, including any range or value therebetween.

In some embodiments, the article of the invention is in a form of a mucoadhesive patch. In some embodiments, the article of the invention is a bio-adhesive flexible patch-like substrate configured for application at a body site. In some embodiments, the article of the invention is for the treatment of a mucus-related disease (e.g. an oral cavity disease).

In some embodiments, the article of the invention is for application on top of a biological tissue. In some embodiments, the biological tissue comprises a mucous tissue, a dermal tissue, or both.

In some embodiments, the article of the invention (e.g. in a form of a patch) is shapeable. In some embodiments, at least one dimension of the article is variable, e.g., by applying stress. In some embodiments, the article is shapeable along at least one dimension, e.g., a length dimension, a width dimension, a radial dimension, a diagonal dimension, and the like. In some embodiments, the article may be shaped and/or elongated, e.g., by a user and/or a medical practitioner, to become elongated, wider, increased in diameter, and/or a combination thereof.

In some embodiments, the article is foldable. In some embodiments, the article is flexible. In some embodiments, the article is characterized by elasticity. In some embodiments, the article is characterized by elasticity and/or foldability sufficient for application of the article on or within one or more region of the tissue (e.g. mucous or dermal tissue).

In some embodiments, the article is configured for application on top or within the biological tissue. In some embodiments, the article is in a form of a patch configured for application on top of the biological tissue. In some embodiments, the article is in a form of an implant configured for application within the biological tissue.

In some embodiments, the nano-particles of the invention are configured to release the encapsulated biologically active agent when the nano-particles are retained inside the polymeric matrix of the invention or from the article comprising thereof. In some embodiments, the nano-particles of the invention are configured to release the encapsulated biologically active agent when the nano-particles are released from the polymeric matrix of the invention or from the article comprising thereof.

The compositions and articles of the invention may further comprise additives such as lubricants, binding agents, excipients, flavors and seasonings. The lubricants used in this invention include, for example, talc, stearic acid and a salt thereof, waxes, etc.; the binding agents include, for example, starches, dextrin, tragacanth, gelatin, hydroxypropyl cellulose, etc.; the excipients include, for example, starches, crystalline cellulose, dextrin, lactose, mannitol, sorbitol, anhydrous calcium phosphate, etc.; and the flavors and seasonings include, for example, citric acid, fumaric acid, tartaric acid, menthol, flavors of citrus fruits, etc. These additives other than the polymers are incorporated in an amount of not more than 40% by weight, preferably not more than 20% by weight, based on the whole weight of the preparation in order to avoid deterioration of the release sustaining properties of the present preparation.

According to another aspect of some embodiments of the present invention, there is provided a kit comprising at least two compartments, such as a first compartment and a second compartment, wherein the first compartment contains the bio-adhesive polymer of the invention; and the second compartment contains the nano-particles of the invention, and wherein the first compartment and the second compartment are substantially dry (having the water content of less than 5%), or in a form of a powderous composition. In some embodiments, the kit comprises the nano-particles encapsulating one or more biologically active agent of the invention.

Alternatively, the kit includes one or more compartments, each containing a pre-measured amount of a dry powder of one or more constituent of the bio adhesive article (e.g. the bio-adhesive polymer and the nano-particles), and optionally a separate compartment containing an additive or the additional layer, as described herein; such that mixing the powder(s) results in the formation of the article, as described herein.

The kit may further include mixing tools, stirring tools, compressing tools, bowls, means for applying the composition at the target site, freshness indicators, tamper-proof measures and printed matter for instructions for the user.

### Methods of Use

The present inventors have shown the feasibility of localized therapy by the administration of the disclosed composition or article comprising thereof.

According to another aspect of some embodiments of the present invention there is provided a method for administering a biologically active agent to a subject, comprising contacting the article of the invention with a biological tissue of a subject, thereby administering the biologically active agent to the subject.

In some embodiments, the biological tissue is as described herein. In some embodiments, the biological tissue is a moist tissue. In some embodiments, the biological tissue is as described herein. In some embodiments, the biological tissue is a substantially dry tissue (e.g. dermal tissue). In some embodiments, the biological tissue is a mucous and/or dermal tissue.

In some embodiments, contacting comprises providing the article and applying the article to a target site of the subject. In some embodiments, applying comprises contacting the bio adhesive surface with the target site (moist or dry) on or within the biological tissue or organ of the subject. In some embodiments, upon contacting the article with the biological tissue, the adhesive surface of the article faces or is bound to the biological tissue. In some embodiments, applying comprises pressing the film towards the biological tissue, so as to induce adhesion of the article thereto.

In some embodiments, applying comprises implanting the article within the biological tissue.

In some embodiments, pressing is by utilizing means for applying the composition at the target site such as an applicator, catheter, or a part of a human body (e.g. hand or finger), etc.

In some embodiments, the article (e.g. a patch) of the invention is a medical device. In some embodiments, the medical device is for use in the field of drug delivery. In some embodiments, the medical device is for administering a pharmaceutically effective amount of the pharmaceutically active agent of the invention to the subject (e.g. locally to a target site on or within the mucous tissue, or systemically via transdermal administration). In some embodiments, the medical device is for administering an effective amount of a biologically active agent to the subject, wherein the biologically active agent is selected from a nutraceutical, or a taste masking agent, a flavoring agent or a combination thereof. In some embodiments, administering comprises local administration and/or systemic administration. In some embodiments, the biologically active agent is as described herein above.

In some embodiments, the method is for topically administering the biologically active agent. In some embodiments, the method is for mucosal or transdermal administration (e.g. oral or nasal administration) of the biologically active agent.

In some embodiments, administering is selected from the group consisting of oral administration, vaginal administration, rectal administration, ocular administration, nasal administration, oral administration, intratracheobronchial administration, pulmonary administration gastrointestinal administration, topical administration and dermal administration, or any combination thereof. In some embodiments, oral administration comprises buccal administration, sublingual administration or both.

In some embodiments, the method is for delivery of the biologically active agent into a mucous or dermal tissue. In some embodiments, the method is for controlled delivery and/or release of the biologically active agent into a mucous or dermal tissue of the subject.

In some embodiments, the method is for transmucosal and/or transdermal administration of the biologically active agent. In some embodiments, the method is for sustained administration of the biologically active agent. In some embodiments, the method is for sustained release of the active agent to the target site. In some embodiments, the method is for sustained release of the biologically active agent to a biological tissue of the subject. In some embodiments, the biological tissue comprises a mucous tissue, a dermal tissue, a muscle tissue, and a urinary bladder tissue or any combination thereof.

In some embodiments, the method is for sustained release of the biologically active agent into an oral cavity and blood circulation. In some embodiments, administration and/or release comprises a pharmaceutically effective amount of the biologically active agent.

According to another aspect of some embodiments of the present invention there is provided a method for preventing or treating a medical condition, comprising administering the article of the invention to a subject, thereby preventing or treating the medical condition, wherein the composition comprises a pharmaceutically effective amount of the pharmaceutically active agent. In some embodiments, administering comprising contacting the article with a biological tissue of the subject, as described herein.

In some embodiments, the method is for reducing and/or ameliorating a symptom associated with the medical condition within the subject (e.g. an oral cavity disease).

In some embodiments, administering comprises oral or nasal administration. In some embodiments, administering comprises topical administration. In some embodiments, administering comprises dermal administration.

In some embodiments, there is provided a method for preparing the article of the invention (e.g. a bio-adhesive article of the invention), the method comprising: exposing an aqueous solution comprising the plurality of nano-particles and the cryoprotectant of the invention to conditions sufficient for drying of the aqueous solution, thereby obtaining a dry powder; mixing a sufficient amount of the dry powder with a powderous composition comprising the bio-adhesive polymer of the invention, thereby obtaining a mixture; and applying a compression force to the mixture thereby manufacturing the article of the invention. Without being limited to any particular theory, the inventors postulated that mixing of the powderous nano-particles with a powderous composition comprising the bio-adhesive polymer is advantageous over lyophilization a solution containing both the bio-adhesive polymer and the nano-particles.

In some embodiments, the method comprises providing an aqueous solution comprising a sufficient amount of: (i) the plurality of nano-particles, (ii) the cryoprotectant and (iii) the bio-adhesive polymer; exposing the aqueous solution to conditions sufficient for drying of the aqueous solution, thereby obtaining a dry powder. In some embodiments, the method further comprises applying a compression force to the dry powder, thereby manufacturing the article of the invention. In some embodiments, the method is for forming a single-layer article, comprising the nano-particles homogenously distributed within the polymeric matrix of the invention.

In some embodiments, there is provided a method for manufacturing a multi-layered article of the invention, the method comprises: (i) applying a compression force to a powderous composition comprising the bio-adhesive polymer of the invention, thereby shaping a polymeric layer; (ii) exposing an aqueous solution comprising the plurality of nano-particles and the cryoprotectant of the invention to conditions sufficient for drying of the aqueous solution, thereby obtaining a dry powder; (iii) applying the dry powder on top of the polymeric layer, thereby obtaining an intermediate layer; and applying an additional polymeric layer on top of the intermediate layer, thereby obtaining a layered composition, and (iv) subsequently applying a compression force to the layered composition, thereby obtaining a multi-layered article of the invention.

In some embodiments, the compression force is sufficient for shaping or molding the article of the invention. In some embodiments, the method comprises a step of molding (e.g. cast molding, compression molding, rotational molding).

In some embodiments, the conditions sufficient for drying comprises lyophilization.

According to an aspect of embodiments of the invention there is provided a medicament comprising one or more compositions or articles disclosed herein, and a pharmaceutically acceptable carrier.

According to some embodiments of the invention, the medicament is being packaged in a packaging material and identified in print, in or on the packaging material, for use in the treatment of a medical condition associated with any disease, medical condition, or disorder as described hereinthroughout.

According to some embodiments, there is provided a method of diagnosing a disease in a subject, the method comprising determining a level and/or activity of at least one saliva secreted marker in a saliva sample of the subject, wherein an alteration in the marker with respect to an unaffected saliva sample is indicative of the disease.

As used herein, the term “diagnosing” refers to determining the presence of a disease, classifying a disease, determining a severity of a disease (grade or stage), monitoring the disease progression, forecasting an outcome of the disease and/or prospects of recovery. In some embodiments, the disease is cancer.

In some embodiments, the disclosed paste or hydrogel further comprises a labeling agent. As used herein, the phrase “labeling agent” or “labeling compound” describes a detectable moiety or a probe. The labeling agent may be attached to a portion of the backbone units forming the polymeric backbone of the paste/hydrogel, directly or via a spacer. Alternatively, the labeling agent may be encapsulated within the void space within the paste/hydrogel.

As used herein, the term “subject” refers to any animal (e.g., a mammal), including, but not limited to, humans, non-human primates, rodents, and the like, to which the compositions and methods of the present invention are administered. In some embodiments, the terms “subject” and “patient” are used interchangeably herein in reference to a human subject. In other embodiments, the terms “subject” and “patient” are used interchangeably herein in reference to a non-human subject.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

General

As used herein the term “about” refers to ±10%.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples which, together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Materials

Sodium Alginate HF120RBS, (molecular weight of 3*105 g mol-1, with G content of around 50%) was kindly supplied by FMC-Biopolymers (Norway). Sodium Chloride (NaCl) were purchased from S. D. Fine-Chem (India) Potassium Bicarbonate (KHCO3) was purchased from Loba Chemie (India). Potassium Chloride (KCl) was obtained from Nile Chemicals (India). Calcium Chloride (CaCl2) was purchased from J. T. Baker (USA). Sodium Phosphate Dibasic (Na2HPO4), Potassium Phosphate Monobasic (KH2PO4), Potassium Thiocyanate (KSCN) and Potassium Thiocyanate (KSCN) were purchased from Merck (Germany). Trehalose, Ethanol (EtOH), Methanol (MeOH) and Acetone were purchased from Bio-lab Ltd. (Israel). Fluorescein Isothiocyanate (FITC), Ethylene Diamine (NH2CH2CH2NH2), 2-(N-morpholino) Ethanesulfonic Acid (MES), N-hydroxysuccineimide (NETS), Sodium Hydroxide (NaOH), Dulbecco's Modified Eagle's Medium (DMEM), Dulbecco's Phosphate Buffered Saline (PBS), Dimethyl Sulfoxide ((CH3)2SO), Isopropyl Alcohol (IPA), Ammonium Sulfate ((NH4)2SO4), Tetrazolium Salt-3-(4,5-dimethylthiazol-2-yl)-2,5-diphenyl Tetrazolium Bromide (MTT), Cholesterol and Sucrose were purchased from Sigma-Aldrich (Israel). 1,2-dimyristoyl-sn-glycero-3-phosphoethanolamine-N-(lissamine rhodamine B sulfonyl) (Ammonium Salt) (14:0 Liss Rhodamine PE) was purchased from Avanti Polar Lipids Inc (USA). HSPC (Hydrogenated Soybean Phosphatidylcholine) and Polyethyleneglycol Distearoyl-phosphoethanolamine (m2000 PEG DSPE) were supplied by Lipoid (Germany). Iron was purchased from Sigma-Aldrich (Israel). 32% Hydrochloric Acid (HCl) and Citric Acid (C6H8O7) were obtained from Frutarom (Israel). 1-Ethyl-3-(3-dimethylaminopropyl)-carbodiimide Hydrochloride (EDC) was purchased from Tzamal D-chem (Israel). Doxorubicin (Dox) was supplied by Teva Pharmaceutical Industries (Israel). L-glutamine, Trypsin, 2-[4-(2, 4, 4-trimethylpentan-2-yl)phenoxy]ethanol (Triton) and Fetal Bovine Serum (FBS), were purchased from Biological Industries (Israel). Cyanoacrylate glue was purchased from Loctite (Israel). Porcine tongue was supplied by the Preclinical Research Authority at the Technion (Israel).

Liposomes Preparation

Liposomes were prepared using ethanol injection method. HSPC, PEG-DSPE and Cholesterol in molar ratio of 55:5:40 were dissolved in absolute ethanol and warmed to 65° C. The lipid suspension was added to 65° C. heated Trehalose solution in DDW (Trehalose:lipids mass ratio of 9:1, DDW:ethanol volume ratio of 9:1), to form multilamellar vesicle (MLV). For Fluorescent labeled liposomes, (1 mg ml-1) 14:0 Liss Rhodamine PE solution at molar ratio of 0.1% with respect to lipid was added to the 10% v/v absolute ethanol. To obtain homogenous nano-particles the mixture of lipids was passed stepwise through polycarbonate extrusion membranes (GE Osmonics, USA), using 400, 200 and 100 pore-size membranes in extruder supplied with a warm bath (Northern Lipids, Vancouver, Canada) at 70° C. Liposome size was determined by Dynamic light scattering (DLS) using a Malvern ZSP. The measurements of particles' size are based on number distribution.

Preparation of Doxorubicin (DOX) Loaded Liposomes

Active liposomes encapsulated with DOX were prepared using an ammonium sulfate gradient. Empty liposomes were made as described above in section 2.2, with (120 mm) Ammonium sulfate solution instead of DDW. The liposome solution was dialyzed against 10% w/w sucrose solution at 12-14 kDa cut-off membrane at 4° C., the dialysis buffer was replaced two times. (2 mg ml⁻¹) of DOX, dissolved in 10% sucrose, was added to the liposomes for 1 hr at 68° C. in a shaker and then placed on ice for 1 min. Dialysis was performed against 10% w/w sucrose solution that was replaced after 1, 4 and 24 hr to remove the external DOX from the liposomes solution. Trehalose in mass ratio of 9:1 (Trehalose:lipids) was added to the solution after dialysis. The amount of encapsulated DOX within the liposomes was determined by measuring the fluorescence using Tecan plate reader (λₑₓ=480 nm, λₑₘ=560 nm), and determining the concentration from a calibration curve of DOX in Ammonium sulfate solution.

Synthesis of Fluorescent Alginate

FITC (1 g) was reacted with an excess of Ethylenediamine (1000 μL) in (10 mL) ETOH for 15 min at room temperature. The solvent was evaporated using rotary evaporation with reduced pressure, then re-dissolved in MeOH and filtrated through a short silica pad to remove traces of Ethylenediamine. The solvent was evaporated under reduced pressure by rotary evaporation, and the orange-red solid was isolated as the final product (primary amine-conjugate to FITC). MES buffer was prepared by dissolving (4.88 g) of MES in (0.5 L) DDW. The pH was adjusted to 6.5 using NaOH. Alginate (1 g) was dissolved in (100 mL) of (50 mm) MES buffer; EDC (780 mg) and NHS (290 mg) were dissolved separately in (20 mL) MES buffer and added to the Alginate solution followed by stirring for 1 hr. Then the primary amine-conjugated FITC was dissolved in (60 mL) MES buffer and added to the Alginate solution. The vail containing the primary amine-conjugated FITC was washed three times with (10 mL) MES buffer (pH=6.5) and the liquid was added to Alginate solution. The mixture was stirred at room temperature for 24 hr, protected from light. Excess of acetone was added to reaction mixture until the FITC-Alginate precipitated. The FITC-Alginate was remove to clean bottle and dissolved with (1 L) DDW overnight. Then the solution was dialyzed against HCL 1% v/v and NaCl 1% w/w for 1 week and during which the dialysis solution was replaced 3 times a day. The final reaction product was freeze dried (Labconco, Kansas, USA) and a yellow-orange powder was separated and stored at 4° C. for future use.

Preparation of Alginate-Liposome Layered Tablet

Liposomes, either empty or DOX loaded, were prepared as described above in section 2.2 and 2.3. Liposomes solution (1 mL) was placed in small tubes and set in liquid nitrogen for two minutes then lyophilized (Labconco, Kansas, USA) at −40° C. and pressure of 0.37 mbar for 24 hr. The liposome size in the dried powder was determined by re-suspending the powder in DDW to the same concentration of the as-prepared liposome solution, and analyzing the size using DLS. The amount of encapsulated DOX within the liposomes was determined from fluorescence measurements as described in section 2.3. Dried alginate was crushed until reaching a fine, uniform powder, and then (40 mg) or (30 mg) was added to a steel mold in the shape of a pill with (1.4 cm) diameter and arranged inside the mold as a uniform layer. (40 mg) of Liposomes', either empty or DOX loaded, was crushed until it formed a fine, uniform powder, and then added to the same steel mold as a uniform layer above the alginate's layer. Finally, (40 mg) or (30 mg) of dried alginate were added as the final layer (alginate constitutes 57% or 43% w/w respectively). The mold containing the layers was compressed using Mega press under high pressure of (250 kg cm⁻²) (Mega, Berriz, Spain). The pill containing the three layers (alginate/liposomes/alginate) was cut to small tablets (1×3 mm) using a sharp knife.

Preparation of Alginate-Liposome Homogenous Tablet

Liposomes, either empty or DOX loaded, were prepared and dried as described above. Dried alginate powder (71%, 57%, 43% and 14% w/w)) was mixed with liposome powder, added to a steel mold, compressed and cut to small tablets as described above.

Preparation of Alginate-Liposome Homogenous and Layered Tablets Containing Iron

Liposomes, either empty or DOX loaded, were prepared and dried as described in section 2.5. 10 mg of iron powder mixed with (60 mg) dried alginate was used in the preparation of layered and homogenous tablet as described above. Iron was utilized as the contrast agent for the CT-imaging.

Preparation of Alginate-Liposome Dry Mixture

Alginate-liposome paste for lyophilization was prepared by dissolving (40 mg) Alginate in (1 mL) of liposome solution, prepared as described above in section 2.2. The mixture was stirred for 12 hr and homogenous viscous solution was attained. The mixture was placed in small tubes and lyophilized as describe above. Liposome size before and after lyophilization was analyzed using DL S.

Liposomes Release Rate from Alginate-Liposome Tablets

Tablets, loaded with labeled liposomes, layered or homogenous, were placed in bottom of a vial and were immersed with (10 mL) simulated saliva buffer (pH=6.8). The vials were placed in a shaking water bath at 37° C. and 25 rpm. At different time points (200 μL) sample was taken from the vial for fluoresces measurements using a Tecan plate reader (λex=530 nm, λem=586 nm) and replaced with fresh simulated saliva buffer. Tablets loaded with empty liposomes were used as a blank for the fluorescence readings. The liposome concentration was compared to a calibration curve of 14:0 Liss Rhodamine PE solution in Simulated saliva buffer (pH=6.8). All measurements were performed in triplicates.

DOX Release Rate from Alginate-Liposome Tablets

Alginate-liposome tablets (1×3 mm) was placed into a dialysis bag and placed in vial with (10 mL) simulated saliva buffer (pH=6.8). These vials were placed in shaking water bath at 37° C. and 25 rpm. An aliquot (200 μL) was withdrawn at different time points and used for measuring the fluorescence (λₑₓ=480 nm, λₑₘ=560 nm) and subsequently DOX concentration from a calibration curve. The sample was then replaced with fresh buffer. Alginate-liposome tablets with empty liposomes were used as blank. All measurements were performed in triplicates.

### Tablets' Adhesion Study

Retention Assay

Evaluation of mucoadhesion properties of the layered and homogenous tablets were accomplished by ascertaining their residence time on porcine tongue mucosa using flow equipment designed as previously described. Frozen porcine's tongue was thawed in 100% humidity and a temperature of 37° C. for 5 min, then a layered or homogenous tablet (1×3 mm), containing Alginate labeled FITC, was placed on the piece of porcine's tissue (2×2 cm) and incubated in the dark in 100% humidity and a temperature of 37° C. for 15 min. Then it was placed on a half pipe, which was anchored on a stand at 45° angle, connected to a channel, syringe and a pumping machine for syringe pump. Small drops of simulated saliva buffer (pH=6.8) were dripped onto the tissue at a constant rate of (6 mL min⁻¹) (total volume of (435 mL)). The drops were collected, accurate volumes of the saliva buffer were measured and a samples of (˜1 mL) in different time points were fluorescently measured using a Tecan plate reader (λem=530 nm, λex=420 nm). Alginate-FITC concentration was calculated according to a calibration curve of the fluorescent Alginate in simulated saliva buffer. All measurements were performed in triplicates.

Tensile Assay

Tensile tests were performed by attaching a small tongue tissue to a specimen holder using 416 Loctite cyanoacrylate glue. The sample holder was then set to the lower arm of a Lloyed Tensile machine equipped with a 50-N load cell. An alginate-liposome tablet with dimensions of 1.4×1.4 cm (prepared as previously described in section 2.6) was glutted to another specimen holder, then fixed to the upper arm of the tensile machine. In order to evaluate the maximum adhesion force (maximum load), the upper arm holding the tablet was compressed against the tongue tissue until a force of (20 N) was recorded and kept for 30 sec. An extension tests were performed, where the upper arm was draw at a constant rate of (1 mm min⁻¹) and the load was measured until detachment was reached. The stress was calculated by dividing the recorded maximum load by the tablet's area. The tests were performed in six independent repetitions.

Cryogenic Scanning Electron Microscope (Cryo-SEM)

Observations of liposomes before and after the lyophilization were carried out using Zeiss Ultra Plus high-resolution SEM, provided with a BalTec VCT100 cold-stage maintained below −145° C. and with a Schottky field-emission electron gun, at an acceleration voltage of 1-3 kV and sample-to-detector distance of 2.5-5 mm. For optimizing high-resolution two of the in-lens type, and two outside the lens detectors were used. A drop of the liposomes' solution (as-prepared or dried and re-suspended) was placed in between two gold planchettes and set within a customized set of tweezers. Next, the tweezers were plunged into liquid nitrogen at −196° C., and the sample transferred into a cooled chamber by liquid nitrogen then into a BAF060 freeze-fracture system (BalTec AG, Liechtenstein) at temperature of −180° C. and a vacuum system. Next, the two gold planchettes were split to fracture the frozen drops. Then 4 nm layer of platinum-carbon at a 90° angle coated the surfaces of the fractured drops. Using cryogenic temperature (BalTec VCT100 shuttle) and a high vacuum the samples were transmitted to the high-resolution scanning electron microscope (HR-SEM) for imaging, which was also cooled in liquid nitrogen.

Scanning Electron Microscope (SEM)

Observations of the solid-state liposomes in the tablet were performed using SEM methodology as previously described in detail. Tablet, prepared as described above, was cut into two pieces and one piece was attached, cut side up, to an SEM stub by a two-sided adhesive tape. The specimen was gold-coated (15 nm layer) in a Polaron E515 sputter-coater. Specimens were evaluate in HR-SEM equipped with a BalTec VCT100 cold-stage maintained below −145° C. and with a Schottky field-emission electron gun, at an acceleration voltage of 1-3 kV.

Fourier Transform Infrared Spectroscopy (FTIR)

Fourier transform infrared spectroscopy (Bruker, Tensor 27, Milan, Italy) was conducted to investigate the interaction between the Trehalose, the biopolymer matrix (Alginate) and the liposomes, before and after lyophilization, using a high-sensitivity LN-cooled MCT detector.

Cell Culture

Murine squamous cell carcinoma cell line, SCC7 purchased from Prof. Reinhard Zeidler, at the German Research Center for Environmental Health in Munich, were cultured in DMEM containing 10% v/v FBS and (2 mM) L-glutamine, kept at a temperature of 37° C. and in humidified atmosphere containing 5% v/v CO2 and 95% v/v air. For maintain cell growth, cells were split every 2-3 days using 6% v/v trypsin solution for detaching.

Cell Viability Assay

Tablets (1×3 mm) containing alginate (0.8, 1.6 mg) and liposomes (0.8 mg) in the form of layered or homogenous, either empty or DOX loaded liposomes were prepared as described above. The final concentration of DOX in each tablet was (1.68 mg mL⁻¹). To examine the toxicity of the tablets containing the drug on SCC7 cell line, at first cells were seeded at a uniform density of 15,000 cells per well on 96 well plates in (200 mL) DMEM medium. On the day of the experiment, medium was replaced with fresh medium containing the tablets, free DOX and in some wells empty fresh medium to serve as control. Cells were incubated for 4, 8, 12, 24, 48 and 72 hr in humidified atmosphere at 37° C. Cells viability was measured using the MTT assay. The medium was removed and (100 μl) of MTT (1 mg mL⁻¹) in PBS⁻¹ were added to each well and incubated at 37° C. for 4 hr. Next, (100 μL) of IPA solution were added to each well, the plates were covered and returned to the incubator at 37° C. for 12 hr. The absorbance was measured at 570 nm and 690 nm for the background absorbance. All measurements were conducted in octuplets.

In Vivo Model Establishment

All in-vivo experiments were conducted upon approval of the ethical committee (Ethical request No. IL-056-05-16). Seven-week-old, C3H/HeJ male and female mice were housed in standard house conditions in sterilized plastic cages in a temperature-controlled room with a 12 hr dark-light cycle and received tap water. Mice were anesthetized with an IP injection of a ketamine-xylazine cocktail ((87.5 mg kg⁻¹) Ketamine, (12.5 mg kg⁻¹) xylazine, to a final volume of (0.1 mL 200) mice) and injected with an analgesic agent Buprenorphine (0.05 mg kg⁻¹) prior to treatment. SCC7 cells ((200,000 cells) per (20 μL) DMEM media) were injected subcutaneously using Terumo® U-100 Insulin 29G×1/2\hat{ }needles to the lateral border of the tongue. Mice were monitored bi-weekly for tumor size and for overall well-being.

Treatment with DOX Liposomal Tablet (Homogeneous and Layered)

Therapeutic treatment was administered twice along the course of 12 d and was initiated when the tumor volume reached 25-50 mm, as measured by a caliper and calculated using the formula V═((width)2×length)/2. Five groups of mice (5 each) were divided to: control—healthy, control—no treatment, empty liposomal layered tablet implant, DOX liposomal layered tablet implants and DOX liposomal homogeneous tablet. Mice were anesthetized with an IP injection of a ketamine-xylazine cocktail ((87.5 mg kg⁻¹) Ketamine, (12.5 mg kg⁻¹) xylazine, to a final volume of (0.1 mL) (200) mice) and injected with an analgesic agent Buprenorphine (0.05 mg kg⁻¹) prior to treatment initiation. Liposomal layers tablet implants were inserted intratumorally through a small incision (2×2 mm) along the lateral border of the tongue with a No. 15 surgical scalpel. The incision closure was achieved by applying Dermabond medical adhesive (Ethicon Inc., Somerville, NJ) through a gentle brushing motion and maintaining manual approximation of the incision edges for 30 sec until full polymerization of the adhesive. Liposomal layered tablet was applied directly to tumor nodule followed by applying (20 ul) PBS above the tablet. After one hour the tablet was removed and the tongue was washed using DDW. At the end of the treatments, mice were sacrificed and tongues were excised and paraffin-embedded for H&E staining.

Computed Tomography

Mice were anesthetized by 1.5% isoflurane and placed on a bed in prone position. For tumor measurements, computed tomography was performed using a small animal imager (IVIS Spectrum CT In Vivo Imaging System, USA). Tumor volumes were obtained by manually delineating margins of tumors from sagittal sections of CT images using Living Image® software.

Statistical Analysis

The data were collected, tabulated and statistically analyzed using the GraphPad Prism v5.01 (GraphPad Software, San Diego, California). All results were expressed as mean±standard deviation (SD). Tumor volume changes before and after treatment was analyzed statistically using paired t-test.

One-way ANOVA was used to analyze the AI of the H&E results between study groups, liposome volume changes, adhesion and release rate results. Also it was used to analyze the MA % and MOD of immunohistochemical results. In all statistical results, a p<0.05 was considered significant. Two-way ANOVA was used to analyze the cell viability results, using different levels of significance p<0.05, p<0.005, p<0.0005 and p<0.0001.

### Example 1

### Effect of Freeze-Drying on Liposome's Properties

### Size and Liposome's Integrity

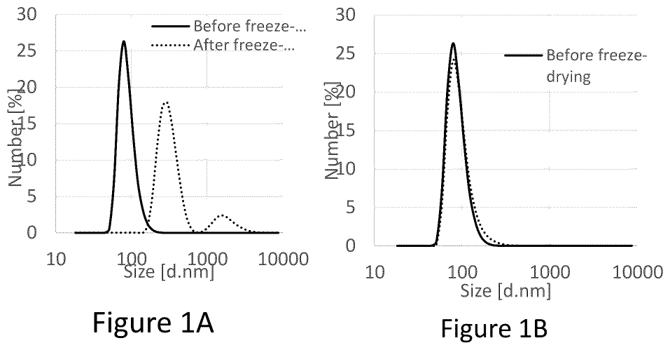

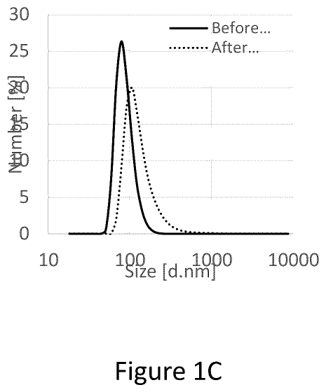

In this research, the inventors aimed to establish a drug delivery system based on drug-loaded lipid nanoparticles in a form of a tablet. In order to provide stability and sustained liposomes release, inventors based the formulation on DOX-loaded liposomal dry powder. An essential step toward achieving this goal was defining appropriate conditions that will inhibit liposome fusion, aggregations and drug leakage during the freeze-drying process. The phospholipids HSPC and DSPE-PEG, and cholesterol, were chosen for the preparation of liposomes. Liposomes containing HSPC are considered to be one of the most stable vesicles, since this lipid preserves the bilayer from outer stresses and tend to show the longest circulation lifetimes. The inventors postulated, that HSPC and DSPE-PEG lipids are expected to protect liposomes from the freeze-drying stress. DLS was used to evaluate the liposomes' particle size, and to detect any differences in size or size distribution, as these are indicative of bilayer disruption and instability (FIG. 1). As expected, lyophilization and rehydration has altered both the average diameter and the size distribution on the liposomes compared to the as-prepared liposomes (FIG. 1A). The monodispersed distribution with an average diameter of 88 nm characterizing the as-prepared liposomes has changed after freeze-drying to a bi-modal distribution with peaks at 307 nm and 1792 nm.

Next, inventors evaluated the effect of lyophilization in the presence of alginate. It was assumed that alginate could be used as a cryoprotectant due to its hydrophilic nature and its ability to create hydrogen bonds. Further, since alginate contributes to mucoadhesion, drying an alginate/vesicles solution could lead to a homogeneous dry mixture that can be directly used to prepare an adhesive, drug-loaded device. Indeed, including alginate in the liposome solution prior to lyophilization has preserved the monodispersed nature of the size distribution (FIG. 1C). The average particle diameter increased from 88 nm to 138 nm, and the distribution width increased from 23 nm to 97 nm. These results indicate that alginate can provide a certain degree of protection against freezing-induced damage.

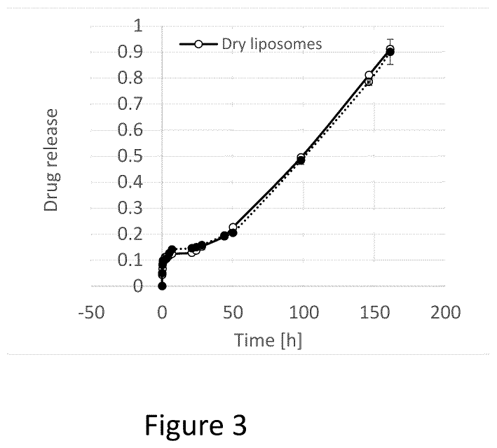

Furthermore, inventors evaluated the influence of a cryoprotectant, trehalose. It was found that the efficiency of size preservation after lyophilization is affected by the amount of added trehalose to the liposome solution. When added at the ratio of 9 w/w, both liposome size distribution and average size are not significantly different than those of liposomes in the initial solution before lyophilization, as shown in FIG. 1B (Anova, p-value=0.9247). These results suggest that trehalose prevented the aggregation and fusion of the liposomes during lyophilization. This suggestion coincides well with the results presented in FIG. 3 that compare between Dox fractional release from liposome solution (before freeze-drying) and powdered liposomes (after freeze-drying) in simulated saliva buffer. No significant changes in the release rate are observed (Anova, p-value=0.8761), thus supporting the suggestion that drug leakage from the dried liposomes is minimal.

### Morphology Evaluation of Liposomes

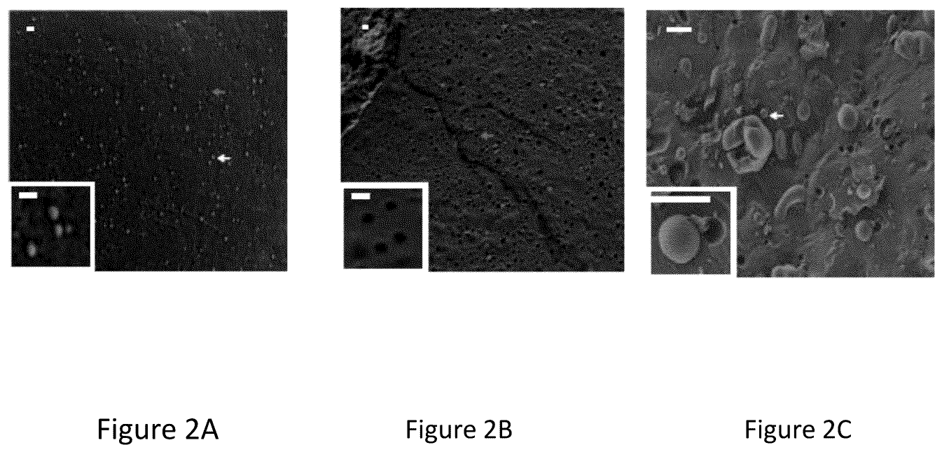

SEM and Cryo-SEM are reliable methods for evaluating liposomes shape and morphology before and after lyophilization and complement the DLS results. Cryo-SEM image of as-prepared extruded liposomes with added trehalose (FIG. 2A) demonstrate smooth surface, spherical shape and homogenous size of about 100 nm. When these liposomes are freeze-dried with trehalose and compressed into small tablet, both liposomes embedded in a matrix of trehalose and their footprints, can be seen in the SEM image (FIG. 2B). The image demonstrates that the size of ca. 100 nm and the spherical shape were preserved. Liposomes upon rehydration after freeze drying without adding trehalose were characterized by an altered morphology in the cryo-SEM (FIG. 2C). Since the shape and the diameter of the liposomes were maintained in the presence of trehalose, despite the compression and the freeze-drying process, there was apparently no phase separation, therefore there is no drug leakage, in agreement with the DLS and drug release results. Such liposomes stability is required for the intended application.

### FTIR Analysis

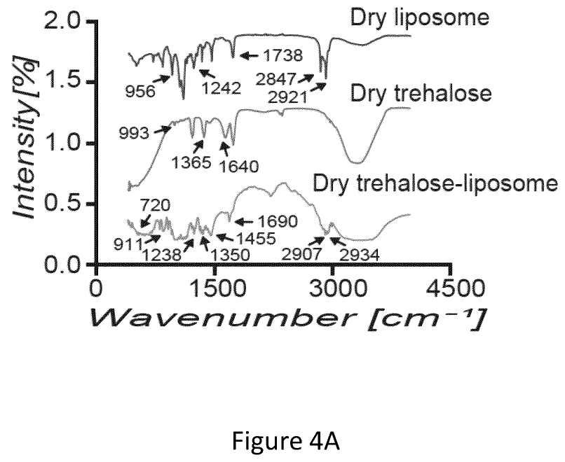

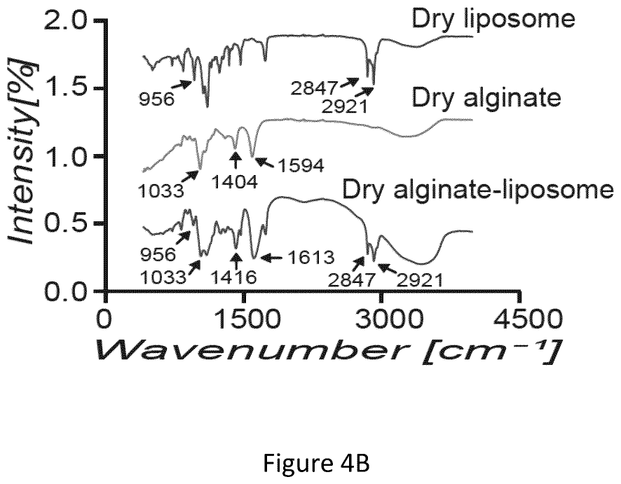

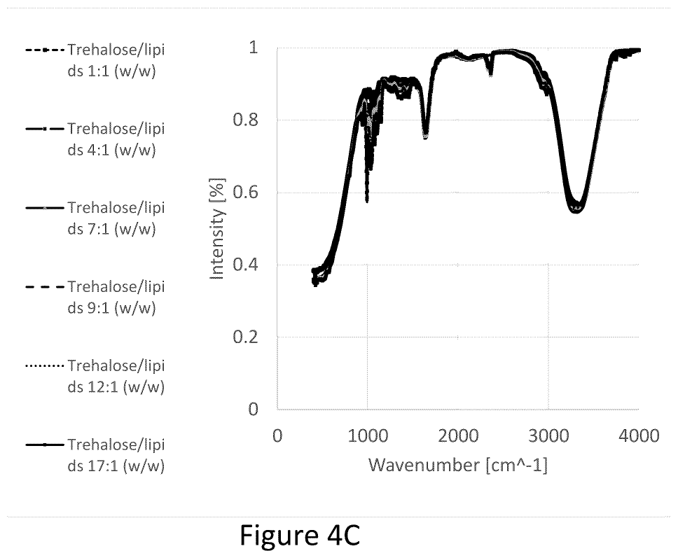

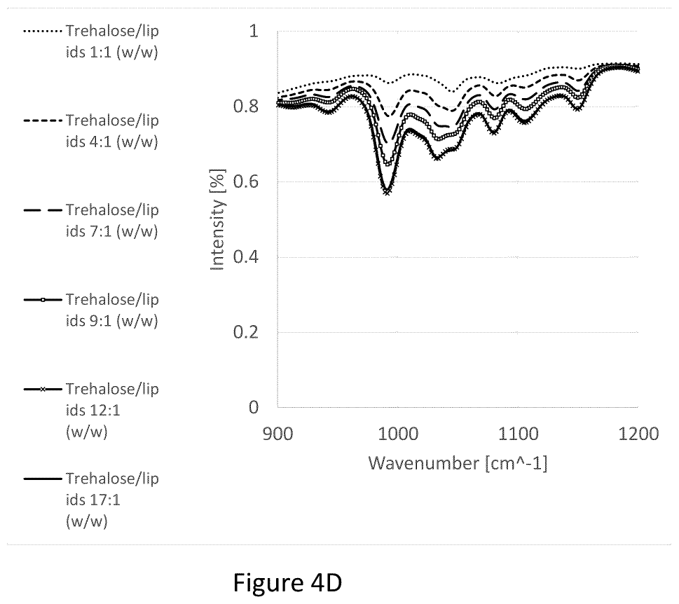

The interactions liposomes with alginate have been investigated using FTIR. The spectra obtained from freeze-dried liposome-alginate mixture is presented in FIG. 4B and compared to spectra from sodium alginate powder and freeze-dried liposomes. The FTIR spectra of freeze-dried liposome-alginate mixture showed a few notable peaks. The characteristic methylene stretching absorption bands at 2921 cm⁻¹ and 2847 cm⁻¹ are due to asymmetric and symmetric CH2 vibration respectively. These peaks also appear at the same location in the spectra of freeze-dried liposomes. The bands corresponding to the asymmetric and symmetric stretching vibration of COO⁻ groups, respectively, shift from 1594 cm⁻¹ for neat alginate to 1613 cm⁻¹ for the alginate/liposome mixture, and from 1404 cm⁻¹ for neat alginate to 1416 cm⁻¹ for the alginate/liposome mixture. These shifts to higher value are indicative of interactions between the liposomes and the COO⁻ groups of alginate, possibly due hydrogen bonds. The positions of the peak at 1033 cm⁻¹ and nearby peaks, corresponding to C—O and C—O—C were the same as the stretching vibration position in alginate spectra. Furthermore, no changes were spotted in the stretching vibration position CN⁺, which also appear in the freeze-dried liposomes spectra at 956 cm⁻¹. The lack of significant changes in the vibrational frequencies peaks at the mixture spectra implies that there is no further interaction via hydrogen bonding between lipids phosphate groups and alginate.

The interactions between trehalose and liposomes were studied by evaluating the vibrational modes of freeze-dried liposomes, trehalose and dry liposomes/trehalose mixture obtained by drying liposome solution containing trehalose (FIG. 4A). The vibrational modes of methylene symmetric and asymmetric stretching, attributed to liposomes, had increased their wavenumber from 2847 cm⁻¹ in neat liposomes to 2907 cm⁻¹ in liposome/trehalose mixture, and from 2921 cm⁻¹ in neat liposomes to 2934 cm⁻¹ in liposome/trehalose mixture, respectively. These peaks are attributed to the hydrophobic part of the liposomes, composed of hydrocarbon chains, and their shifts may occur due to the freeze-drying process or the formation of new bonds with other parts in the liposomes. The spectrum from freeze-dried liposomes shows additional peaks at 1738 cm⁻¹ and 1242 cm⁻¹ corresponding to C═O stretching and PO₂ asymmetric stretching. These bands shifted towards lower frequencies in the presence of trehalose, which suggests hydrogen bonding between the phosphate headgroups of the liposomes and the OH groups of the trehalose. In the case of choline vibrational modes, attributes to HSPC lipid which is one of the main components in the liposome bilayer, the peak 956 cm⁻¹ attributed to CN⁺ shifted toward lower wavenumbers 911 cm⁻¹. This shift also points to bonding establishment.

The spectra of trehalose powder show a peak at 1640 cm⁻¹ attributed to H—O—H bending motion, corresponding to the bending vibration of residual water. In the freeze-dried liposomes/trehalose powder the wavenumber shifted to lower value, probable due to phase change of remained water molecules adsorbed on the surface of the sample. O—H bending peak appears at the trehalose FTIR spectra at 1365 cm⁻¹ and in the liposome/trehalose spectra the peak appears at a lower value of 1350 cm⁻¹, due to hydrogen bond formation, in agreement with previous observations. The peaks at 993 cm⁻¹ attributed to symmetric stretching modes of the bridge C—O—C in the α-(1-1)-glycosidic bond shift to around 720 cm⁻¹ due to interactions of the sugar with other molecules.

Overall, the multiple shifts observed following trehalose addition are indicative of hydrogen bonds formation between the sugar and the polar groups on the nanoparticles surface of the, replacing water molecules and immobilization, which is a well-known mechanism for preserving the primary structure of nanoparticles in the freeze-drying process. As stated above, DLS results indicated that the sugar concentration affects the susceptibility of liposomes to the freeze-drying process. At a mass ratio of 9:1 (trehalose:lipids) a substantial size preservation was obtained. FIG. 4C shows the FTIR spectrums of different amount of trehalose added to liposomes solution before freeze-drying. These spectrums explicitly present the evolution of the peak at 1044 cm⁻¹ attributed to C—O—C in the α-(1-1)-glycosidic bond of trehalose to lower frequency as the amount of added trehalose is increased. This shift is clearly larger when the trehalose:lipids ratio is 9:1 or higher. Apparently, the addition of trehalose facilities water displacement and formation of new bonds between the glycosidic bond zones and the liposomes that allow liposome stabilization.

### Example 2

### Effect of Alginate on Release Rate and Stability

An essential design parameter is the ability to release most of the drug gradually in a rate required to reduce the tumor's size. It was hypothesized that a dry compressed tablet composed of dry alginate and dry liposome powder could provide a desired slow-release characteristic, since its dissolutions is a prerequisite for liposomes release. Further, it was assumed that tablet dissolution rate and the resulting release rate will depend on both the amount of alginate and its distribution relative to liposomes within the tablet. Two means of distributing the alginate in the tablets were investigated. The first was homogeneous distribution (FIG. 6B), achieved by compressing a mixture of dry alginate powder and dry liposome powder. The second is layered “sandwich-like” arrangement (FIG. 6A), obtained by arranging a layer of dry alginate inside the mold, placing a second layer of dry liposome powder on top of it, covering with a second alginate layer, and compressing. The two types of tablets were investigated in order to characterize the effect of alginate amount and its distribution on the liposome/drug release rate. Tablets were prepared from liposome labeled with rhodamine florescence dye, and loaded with doxorubicin, a clinically approved chemotherapeutic drug characterized by strong fluorescence. This approach allowed a detection of both liposomes and drug in the release medium.

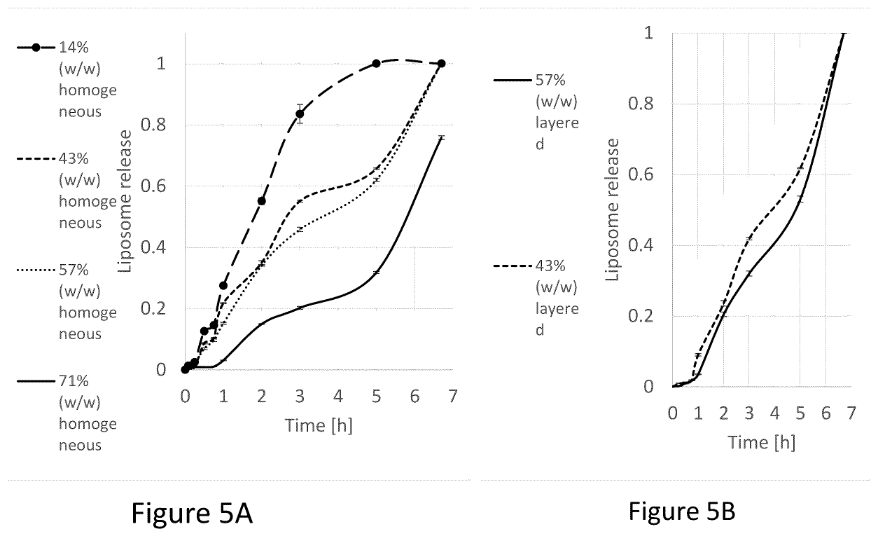

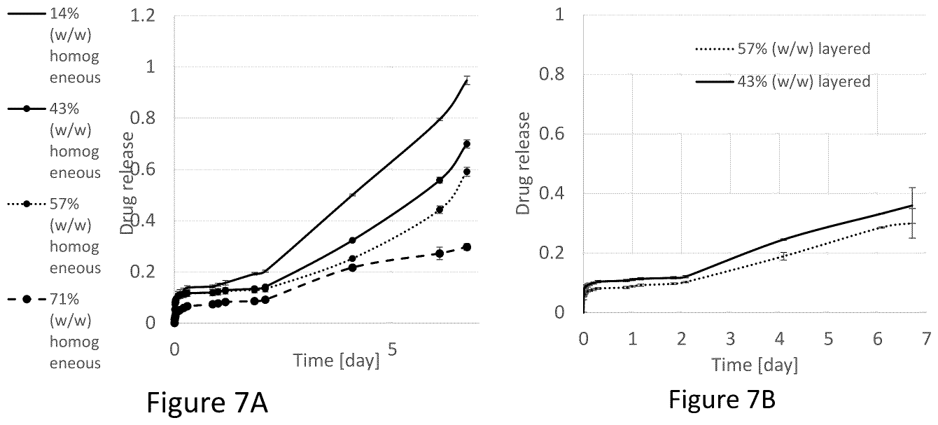

The release rate of liposomes was studied by submerging a tablet in simulated saliva buffer and detecting the signal from the labeled liposomes. Under these conditions, the buffer diffuses into the polymeric matrix of the tablet, resulting in gradual dissolution of the polymeric network and enabling liposomes sustainable release. FIGS. 5A and 5B show fractional release of liposomes from the two types of alginate-liposome tablets, homogeneous tablets and layered tablets, respectively. For both types of tablets a short lag time of approximately 1 h is noticed, followed by a slow and moderated liposomes release. With the exception of the homogenous tablet with the lowest alginate percentage, a second sustained release phase with higher rate is then observed. Full release was obtained from all tablets after 7 h except of the one containing 71% (w w⁻¹) alginate. These results imply that three distinct stages are involved in the release mechanism. First, the aqueous solution diffuses through the surface layer of the tablet forming a gelatinous layer while inside the tablet remains dry. The lag period is the time passing until the hydrated layer contains enough water to allow liposome transport. At longer times, the hydrated outer layer determines the diffusion of the solution into the polymer matrix and inner liposome core resulting in dissolution the outer network alginate and a liposomes release. In general, as represented in FIGS. 5 and 7, when the amount of alginate increases in the same tablet type, the liposomes release rate slowed down, presenting prolonged and controllable release. This effect is statistically significant (Anova, p-value=0.0002 and p-value=0.0439 for FIGS. 5A and 5B respectively). For example, during the first 5 h, the release from the homogenous tablets containing 71% (w w⁻¹) alginate reached ˜75% of the initial amount of liposomes, a much lower value than homogenous tablets containing 14% (w w⁻¹) alginate, which permitted a fully release of the liposomes. These results could be due the high polymer concentration which results in forming a thick resistant gel matrix barrier with high viscosity that reduces liposomes diffusion coefficient and thus the release rate. From a technological point of view, varying alginate content can be used to control the release rate and adapted it to the oral mucosal epithelium condition.

Comparing the release of liposomes from homogeneous tablets (FIG. 5A) to that from layered tablets (FIG. 5B) indicates that the release from layered tablets with the same alginate content lower. This effect can be attributed differences in alginate and liposomes dispersion in the tablet. In the layered tablets, the alginate has to be fully dissolved before liposomes can diffuse out from the tablet. Further, since all alginate is located near the tablet's boundaries, it forms thick and dense layer which slows down liposome diffusion. On the contrary, in homogenous tablets liposomes are evenly dispersed hence are also located near the tablet's edge. Alginate dissolution begins faster, and water entering the tablet further enhances alginate dissolution and liposomes release rate.

In order to evaluate Dox release rate, the tablets were placed into dialysis bag immersed in simulated saliva buffer. Only the drug can pass through the bag and be detected in the outer buffer. FIGS. 7A and 7B display the fractional release of Dox from homogenous and layered tablets with diverse amount of alginate. It can be observed that drug release rate increases with decreasing alginate content. The influence of alginate content is in line with the mechanism described above, and is statistically significant (Anova, p-value<0.0001 and p-value=0.0038 for FIGS. 7A and 7B respectively). However, the release rate of doxorubicin is much slower than the liposomes release rate, resulting in release of less than 15% of the loaded dox in 24 h and less than 50% in 98 h from tablets containing 14% (w w⁻¹) alginate. About 90% of Dox was released in approximate 7 days while the value was less than 70% for the other tablets. These results can be attributed to the high stability of drug-loaded liposomes over time. When buffer solution diffuses to the alginate matrix making it prone to erosion, the liposomes are released within few hours. However, doxorubicin remains trapped within the liposomes for days and released only when there is phase separation or other disruption of liposome stability leading to drug leakage. It is noted that in vivo drug can be also adsorbed by liposome uptake.

Furthermore, the inventor successfully implemented similar dry matrices based on chitosan as the bio adhesive polymer. Exemplary dry (e.g. lyophilized) compositions including liposomes embedded within the chitosan matrix and trehalose as a cryoprotectant, have been successfully implemented by the inventors and currently undergo additional studies in order to evaluate drug release profile.

### Example 3

### Adhesion Assays

### Tensile Study

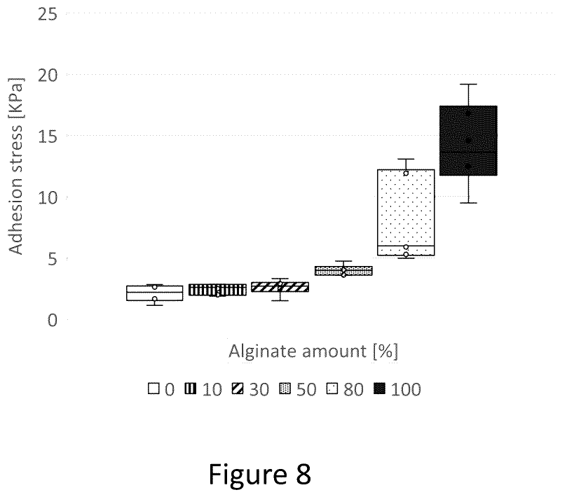

In order to allow long-term topical treatment, the drug-containing tablets should display mucoadhesive properties, i.e. adhere to the oral mucosa hence providing enough time for drug uptake by the tissue. In this study, we used a tensile machine to evaluate the adhesion properties of tablets with different alginate content (FIG. 8). The stress at maximum load significantly increased with the amounts of alginate (Anova, p-value<0.0001). However, when the alginate content was higher than 30%, no further significant effect on the stress was noticed.

### Retention Study

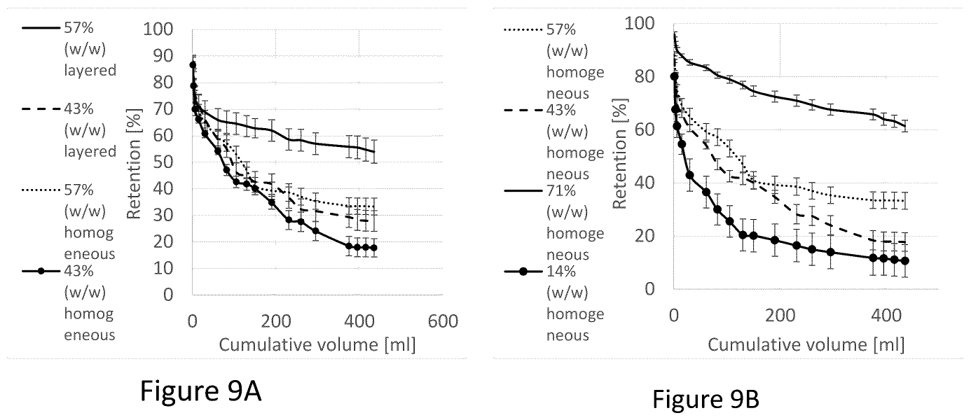

In order to obtain the effect of changing small amounts of alginate powder in tablets on the adhesion properties, a more sensitive method was used to evaluate the alginate mucoadhesive features, a flow through experiment. FIGS. 9A and 9B display the adhesion properties of different tablets with diverse alginate amounts. FIG. 9B demonstrates that the mucoadhesion properties of tablets with homogeneous distribution of alginate significantly decreases with a decrease in alginate content (Anova, p-value<0.0001). Tablet with the highest initial alginate amount, 71% (w w⁻¹), revealed the highest retention at short times, reached a steady state value of 61% retention after washing with 435 ml of simulated saliva buffer. The lowest retention of ˜9% after washing with 435 ml buffer was observed for tablets with 14% (w w⁻¹) alginate.

As for the adhesion properties of the layered tablets, the retention of the tablet with 57% (w w⁻¹) alginate decreased and reached a steady state at ˜54% after washing with 435 ml of simulated saliva buffer (FIG. 9A). The tablet with the lower alginate content of 43% (w w⁻¹) showed significantly faster clearance (Anova, p-value<0.0001) and lower final value of ˜28%. Thus, for both homogeneous and layered tablets, the adhesion performance of the tablet increases with alginate content, indicating that the mucoadhesive properties can be attributed to alginate.

Moreover, a comparison between the adhesion performance of homogeneous tablets and layered tablets (FIG. 9A) showed that the layer tablet displays higher retention than homogenous tablet with the same alginate amount (Anova, p-value<0.0001 and p-value=0.0190 for 57% and 43% (w w⁻¹) respectively). It is postulated that a stronger bonding with the mucosal tissue might be formed, when a uniform alginate layer is utilized in an exemplary article of the invention. It is postulated that alginate layer in contact with the tissue facilitates the formation of stable bonds therebetween. It is postulated, that in the case of the homogenous tablet, part of the tablet's surface contacting the tissue is composed of liposomes that have relatively low adhesion. In view of the results, in order to obtain a desirable and effective adhesive tablet tailored to our application goals, it should contain sufficient alginate amount to get the optimal adhesion properties and to enable liposomes release therefrom.

### Example 4

### Cells Viability

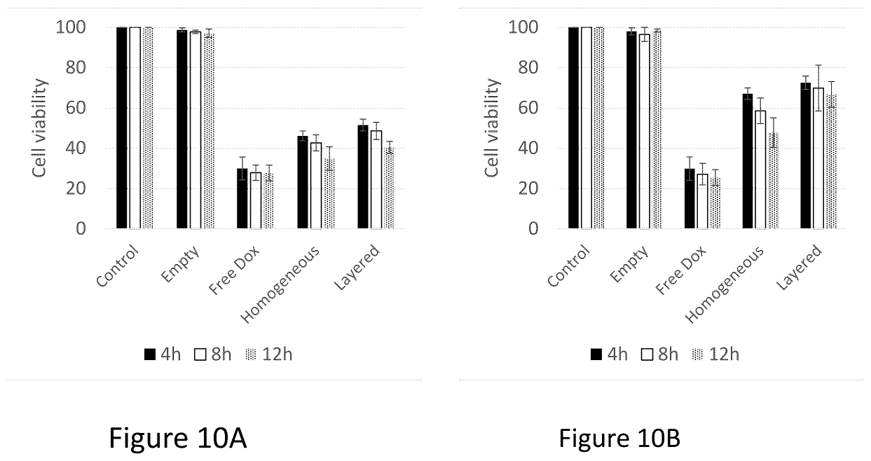

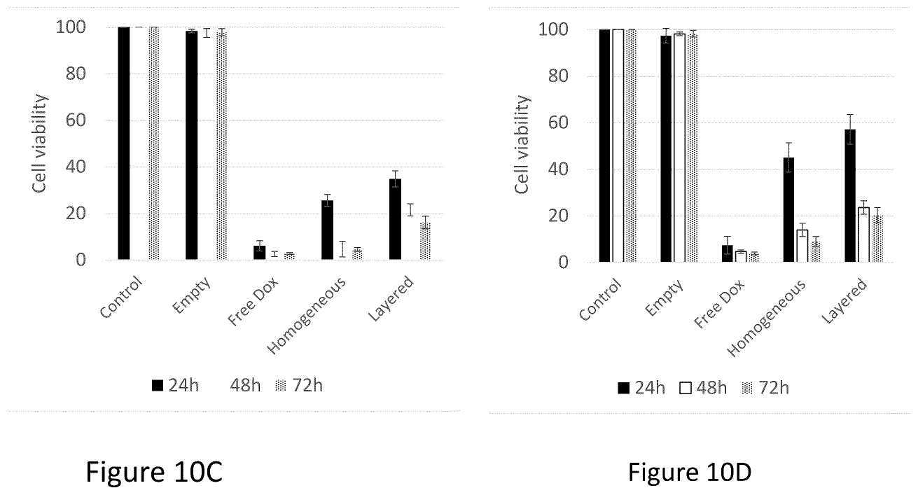

The short- and long-term toxicity of the drug loaded liposome-alginate tablets (homogenous and layered) was investigated on murine squamous cell carcinoma cell line SCC7(FIG. 10). The results were compared with those attained using free DOX and a tablet with empty liposomes. A tablet comprising empty liposomes had no effect on the cell viability for both short and long times, while free DOX was the most effective in decreasing cell viability to 3-10% and 28-40% at long and short times respectively. Tablets with Dox loaded liposomes displayed a dose-dependent decrease in cell viability, which could be further controlled by using layered or homogenous tablets. After only 4 hr of incubation, cell viability declined to −72% and 68% for homogeneous and layered tablets containing 57% (w w⁻¹) alginate, respectively. Cell viabilities for homogeneous and layered tablets containing 43% (w w⁻¹) alginate were significantly lower, ˜52% and ˜46% respectively (Anova, p-value<0.0001 for all tablets). The lower viabilities of cells incubated with tables with 43% (w w⁻¹) alginate can be attributed to larger Dox release from the tables described above (FIGS. 7A and 7B). The results presented in FIG. 10 further indicate that the longer the tablets are exposed to cells, the higher the cancer cell death (Anova, p-value<0.0001 for long and short term). The data revels lower cell viability in homogenous tablets compared to the layered tablet, which can be attributed to the faster DOX loaded liposomes and higher cell uptake. These observations are consistent with our previous observations and strengthen the suggestion that the liposomes released from tablets maintained stable and effective despite the freeze-drying process.

### Establishment of Squamous Cell Carcinoma Model and Treatment Modalities

To establish a squamous cell carcinoma mouse model, 2*10⁵ SCC⁻⁷ cells were directly inoculated to the lateral side of the tongue. Tumor nodules were noticed after approximately 3 days post inoculation and ranged from endophytic ulcers to exophytic masses. Tumor size reached 27-40 mm³ after 7 days, also H&E histology evaluation further demonstrates well differentiated squamous cell carcinoma in cancer induced mice, whereas healthy mice showed normal tongue histology with sharp conical projection of filiform papillae with thin smoothed keratinized epithelial covering.

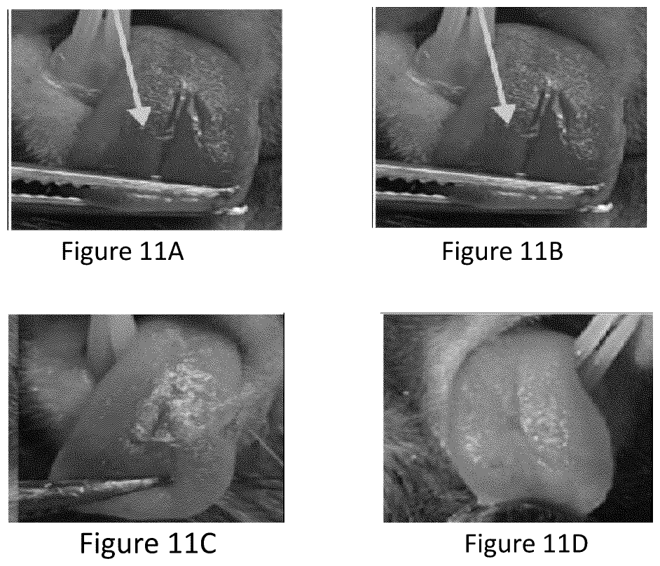

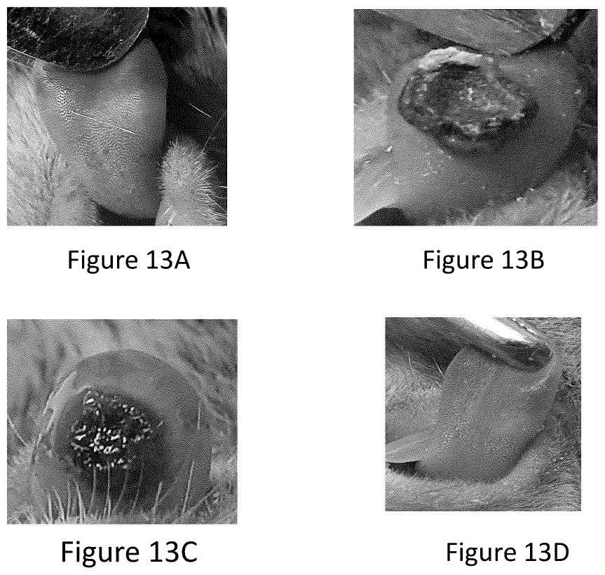

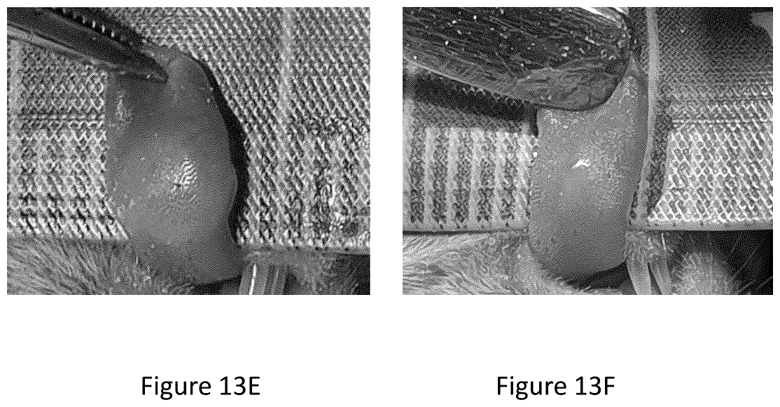

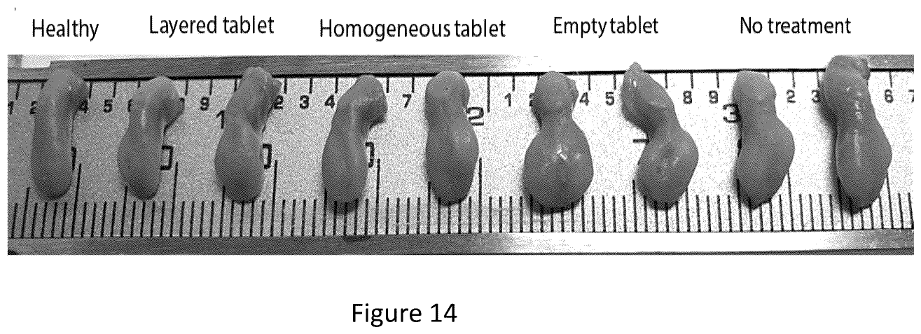

For developing the surgical protocol, the liposomal layered tablet were inserted into the tongue by making a small incision to create a pocket for the implant and after implant insertion, the incision was sealed using a medical adhesive. 24 hours post insertion, the wound was completely healed, the animals were able to eat crumbled food, acting normally and showed no sign of distress (FIGS. 11A-D). As for the liposomal layered tablet is placed above the protruded tumor on tongue for 1 hr and no further surgical maneuvers were needed, also animals well-being was not affected by this treatment modality (FIGS. 13A-D).

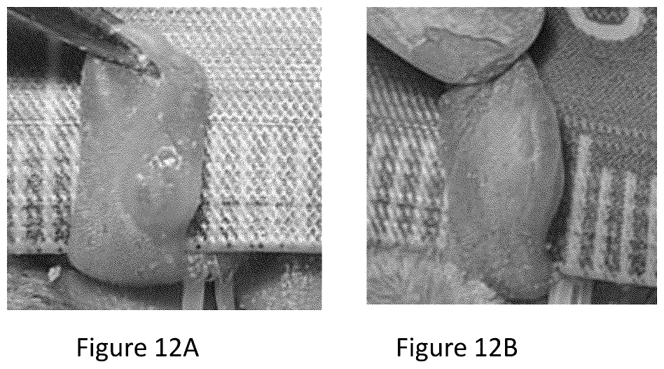

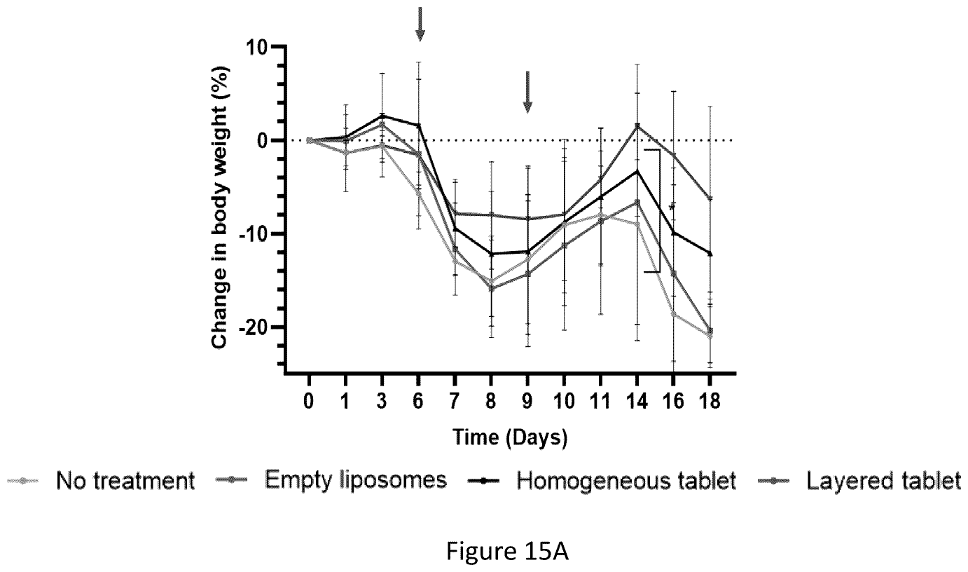

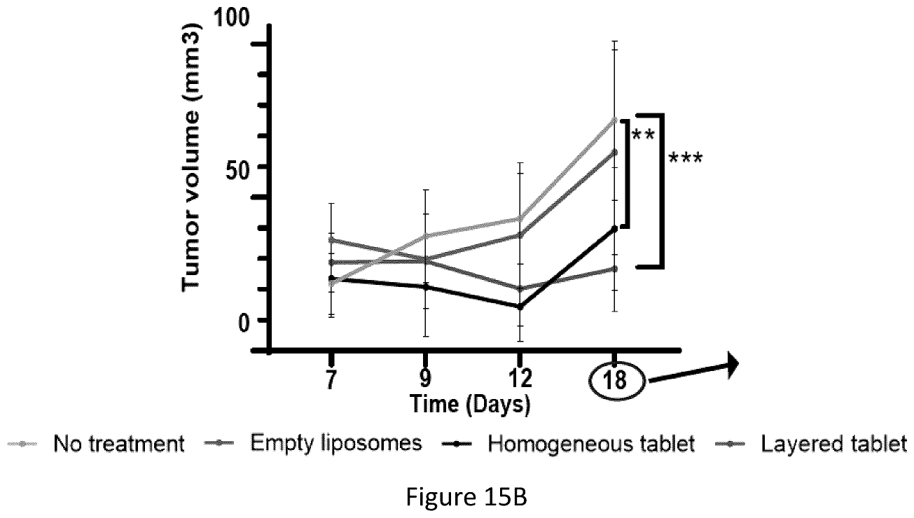

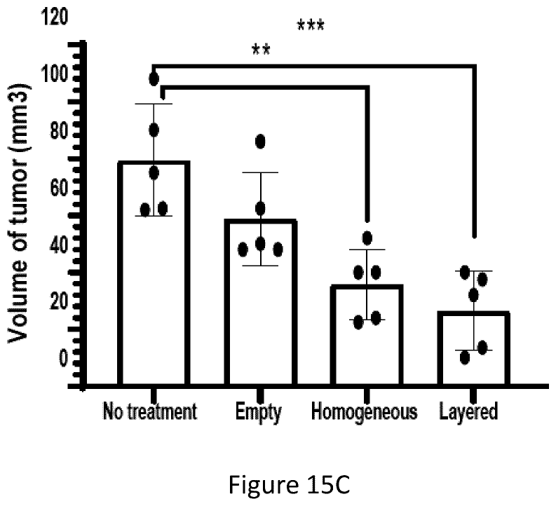

Clinical Evaluation of the Proposed Treatments For testing treatment efficacy, animals were randomly divided into five groups (n=5 each group), according to the type of proposed treatment; DOX liposomal tablet homogeneous implant, empty-liposomal homogeneous tablet implant, DOX liposomal layered tablet. The tumor volumes in all groups were recorded from the cancer induction until animal sacrifice. Furthermore, the animals' body weight as well as their overall well-being was monitored daily. From representative images of the tumor volume following 12 days of treatment (layered/homogeneous tablet) (FIGS. 15A and 15B). demonstrate that, the tumor volume in both treatment groups were evidently lower than that of non-treated group. Tumor volume measurements indicate a decrease in tumor growth in mice receiving both liposomal homogeneous tablet implant and layered tablet in comparison to the healthy control and empty liposome treatment (FIGS. 12, 13E-F, 15C). It is worth noting that there was some reduction in tumor volume in the empty liposomal tablet implant in comparison to non-treatment mice (59±14 mm 3 FIGS. 15B, 15C), this may be explained by the incision made in the tumor and the disturbance of cancer cells, also wound healing has been shown to promote transformation of malignant cells through the infiltration of immune cells which eventually reduce tumor size.

After 18 days of cancer induction, the average tumor size of DOX liposomal homogeneous tablet implant and layers tablet was 36±11 and 27±12 mm 3 respectively, in comparison to non-treated mice that showed a size of (80±18 mm 3) (FIG. 15B, 15C). Concisely, the examined tumor volumes showed a significant reduction of 55% and 67% compared to non-treated animals (p<0.01 for DOX liposomal homogeneous tablet implant and p<0.001 for DOX liposomal layered tablet). The overall body weight monitoring of animals further elucidates the wellbeing of mice in the different group, indicating a significant difference in body weight reduction between mice treated both with dox liposomal-tablet layers (7±2%) compared to non-treated and empty liposomal tablet (23±4%, 24±5% respectively) (FIG. 15A).

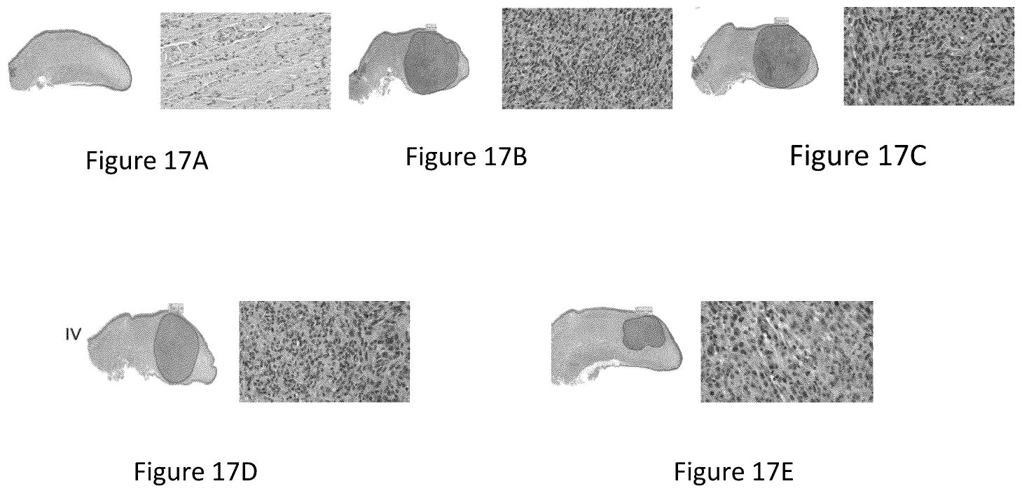

Thorough histological evaluation was performed by a qualified pathologist. The H&E stained tongue tissue sections were assessed and classified as squamous cell carcinoma, hyperplasia, or normal per animal. Normal oral mucosa of the tongue squamous epithelium was observed in tongue tissues of the normal group animals (FIG. 17). Treated groups' histological images revealed a significant decrease in the malignancy grade of the groups receiving both DOX liposomal homogeneous tablet implant and layer tablet, furthermore, histology slides clearly show tumor area shrinkage in treated groups in comparison to non-treated (1.7 folds for DOX liposomal homogeneous tablet implant and 3.9 folds for DOX liposomal layered tablet) and empty-liposomal tablet implant treated group (1.6 folds for DOX liposomal homogeneous tablet implant and 3.5 folds for DOX liposomal layered tablet) (FIG. 17).

To further elucidate the effect of the local treatment, mice were subjected to computer tomography (CT) imaging, processing axial, sagittal and coronal view, immediately after treatment initiation and at the end of the treatments course.

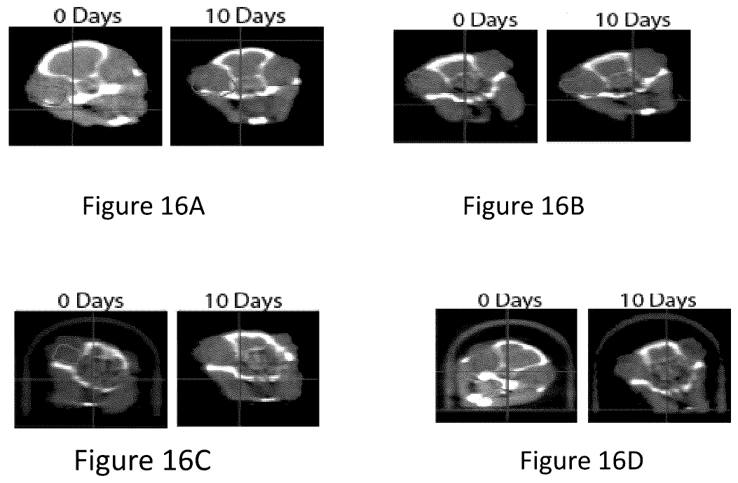

The CT images demonstrate the inhibitive effect of the drug delivery system on the tumor cells proliferation ability. Untreated mice demonstrate 4 folds increase in the tumor growing area compared to treated mice with the drug delivery system. Untreated mice also present a very advanced disease with ulcer formation and bone invasion (FIG. 16). Interestingly, mice that were treated with DOX liposomal-tablet implants presented a relatively inferior inhibitive effect when compared to DOX liposomal homogenous tablet. In DOX liposomal homogeneous tablet group the tumor margins were more confined 12 days post treatment, however, no tumor shrinkage was seen on the other hand, mice treated with DOX liposomal layered tablet demonstrated an impressive tumor shrinkage, confined and non-invasive tumor.

To summarize, the experiments demonstrate successful in-vivo treatment of oral squamous cell carcinoma (OSCC) tumors using DOX liposomal homogeneous tablet implant or layered tablet. Both treatments had a positive outcome and inhibited the tumor growth, with reduction in tumor volume. The histological and CT scans further proved the cytotoxic effect of the proposed treatment modalities in the treatment of OSCC, by decreased density of dysplasia and squamous cell carcinoma together with tumor area shrinkage as observed in CT scans. In OSCC, DOX exhibits high tumor-specific cytotoxic activity. It was found to induce apoptosis through the activation of caspase-3, -8 and -9 in vitro in OSCC cell line (HSC-2). However, the major limitation for the systemic in-vivo use of DOX is the reported cardiotoxicity. In the current study the inventors introduces two liposomes-based treatment modalities for local chemotherapy of OSCC. The small size of the nanoparticles reduces the total administered amount of the loaded drug, while preserving its therapeutic effectiveness. In addition, the ability of liposomes to penetrate the capillaries increase the concentration of the chemotherapeutic agent in the cancer cells. Moreover, the superior anti-tumor effects of targeting the tumor site with the chemotherapeutic drugs through direct delivery encourage the ongoing studies in the field of local chemotherapy.

To this end, the inventors successfully developed and characterized innovative controlled drug release system providing good stability, sustained release and adhesion performance. Liposomes showed high stability, minimal drug leakage and efficiency in the presence of trehalose, during the freeze-drying process. This dry formula provides multiple advantages over liquid formulars by having longer shelf life and exact dosages. In addition to a sustained release rate by using different forms of tablets and varying amounts of alginate, due to the degradation rate of the polymeric matrix. Moreover, in-vivo results show that alginate doxil tablet inhibits tumor growth and can potentially be a clinical therapeutic strategy for oral cancer.

Additional data together with non-limiting exemplary representations are disclosed in the Appendix incorporated herein by reference.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition comprising a polymeric matrix and a plurality of nano-particles embedded within the polymeric matrix, wherein:
(a) said polymeric matrix comprises a bio-adhesive polymer;
(b) said plurality of nano-particles encapsulate a biologically active agent,
(c) said composition comprises a cryoprotectant;
(d) a water content of said composition is at most 5% by weight; and wherein a weight per weight (w/w) concentration of said a bio-adhesive polymer within said composition is at least 10%.

2. The composition of claim 1, wherein at least 99.5% w/w of said bio-adhesive polymer is not-crosslinked; wherein said polymeric matrix is characterized by a network of interconnected chains of said bio-adhesive polymer and wherein a w/w concentration of said a bio-adhesive polymer within said composition is at most 95% w/w; wherein said polymeric matrix is in a form of a layer, and wherein said plurality of nano-particles are homogenously distributed within said layer.

3. (canceled)

4. (canceled)

5. The composition of claim, wherein a w/w ratio of said cryoprotectant to said plurality of nano-particles is between 2:1 and 20:1.

6. The composition of claim 1, wherein a w/w ratio between said bio-adhesive polymer and said plurality of nano-particles is between 10:1 and 1:10.

7. (canceled)

8. The composition of claim 1, wherein said composition comprises a plurality of layers; wherein said composition comprises an upper layer and a bottom layer comprising said polymeric matrix; and further comprises an intermediate layer comprising said plurality of nano-particles.

9. (canceled)

10. The composition of claim 1, wherein said bio-adhesive polymer comprises a mucoadhesive polymer selected from the group consisting of: alginate, chitosan, pectin, hyaluronic acid, PVA, and polyacrylate, including any salt, derivative, copolymer, or any combination thereof.

11. The composition of claim 1, wherein said plurality of nano-particles comprises a lipid and are in a form of liposomes or micelles wherein said lipid comprises a phospholipid and a sterol; optionally wherein said sterol comprises cholesterol, and said phospholipid comprises a zwitterionic lipid, an anionic lipid, a PEG-ylated lipid including any combination thereof.

12. (canceled)

13. (canceled)

14. The composition of claim 11, wherein a w/w ratio between said phospholipid and said sterol is between 1:4 and 4:1.

15. The composition of claim 1, wherein at least 80% of said plurality of nano-particles have an average diameter of between 50 and 500 nanometers; and wherein said plurality of nano-particles is characterized by a polydispersity index of 0.03-0.3.

16. (canceled)

17. The composition of claim 1, wherein the molar concentration of the said plurality of nano-particles within said composition is about 10-200 millimolar (mM).

18. The composition of claim 1, wherein said cryoprotectant is selected from the group consisting of a disaccharide, DMSO, a glycol, glycerol, or any combination thereof; and wherein a w/w concentration of said cryoprotectant within said composition is between 0.1 and 10%.

19. (canceled)

20. The composition of claim 1, wherein upon reconstitution of said composition the average diameter of said plurality of nano-particles increases by at most 30%; wherein upon contact with a mucous tissue said composition is capable of releasing at least 50% of said biologically active agent within a time period of between 0.1 and 24 h.

21. (canceled)

22. (canceled)

23. An article comprising the composition of claim 1, and wherein said article is a bio-adhesive article, optionally characterized by mucoadhesiveness.

24. The article of claim 23, comprising an effective amount of the biologically active agent, wherein the biologically active agent comprises any one of: a pharmaceutical agent, a nutraceutical agent, a taste masking agent, a flavoring agent optionally wherein said article is in a form of a film, and wherein said article further comprises an additional non-mucoadhesive layer; wherein a thickness of said article is between 0.1 and 10 mm, and any one of a length dimension and of a width dimension of said article is between 1 mm and 10 cm: wherein said mucoadhesiveness comprises stress at maximum load of at least 2 KPa.

25. (canceled)

26. (canceled)

27. The article of claim 23, wherein said biologically active agent is characterized by having a therapeutic effect in the treatment of an oral cavity disease.

28. (canceled)

29. A method for preventing or treating a medical condition, comprising administering the article of claim 23 to a subject, thereby preventing or treating said medical condition; wherein said administering comprising contacting the composition with a biological tissue of said subject.

30. (canceled)

31. The method of claim 29, wherein said biological tissue comprises a mucous tissue, a dermal tissue, a muscle tissue, and a urinary bladder tissue or any combination thereof.

32. The method of claim 29, wherein said administering is selected from the group consisting of oral administration, nasal administration, and dermal administration, or any combination thereof; optionally wherein said oral administration comprises buccal administration, sublingual administration or both; and wherein said medical condition comprises an oral cavity disorder.

33. (canceled)

34. (canceled)

35. A method of manufacturing the article of claim 23, comprising:
a. exposing an aqueous solution comprising the plurality of nano-particles and the cryoprotectant to conditions sufficient for drying of said aqueous solution, thereby obtaining a dry powder;
b. performing any one of (i) or (ii):
(i) mixing a sufficient amount of said dry powder with a powderous composition comprising said muco-adhesive polymer, thereby obtaining a mixture;
(ii) applying a compression force to a powderous composition comprising a bio-adhesive polymer, thereby shaping a polymeric layer; applying said dry powder on top of said polymeric layer, thereby obtaining an intermediate layer; and applying said polymeric layer on top of said intermediate layer, thereby obtaining a layered composition; and
c. applying a compression force to said mixture or to said layered composition, thereby manufacturing said article.

36. The method of claim 35, wherein said conditions sufficient for drying comprises lyophilization; optionally wherein said bio-adhesive polymer comprises a mucoadhesive polymer selected from the group consisting of: alginate, chitosan, pectin, hyaluronic acid, PVA, and polyacrylate, including any salt, derivative, copolymer, or any combination thereof.

37. (canceled)
